SEQUENCE LISTING (23 sequences)

SEQ ID NO: 1 - DNA, 1431 bp, Halomonas sp
  CDS (1)..(1431)
  sig_peptide (1)..(78)
  mat_peptide (79)..(1428)
atgcgtccgattactcgttcttttcctttgtgggctttacttttagctcttcttcttccttggcaagctgcgttggctcgcgaccttccagattttgctaaccttgtagaacaagctgctcctggcgtagttaacatttctactactcatacagttgaacgcggtcaggcattcgagggctttggcggacaagatattccagacattttccgtcatttcttcggtgaccgtttccctggtggacctgaaggtccaatgccaggacctggaccaggcccaagccaagagcgccaatctcttggttcaggctttatcatctctgaagatggttacattcttacgtctgcgcacgtagtagaaggtgcggatgaaatccttgttcgtttagcggatcgtcgtgaaatggaagctgagcttgttggagcagacatccagactgacgttgcagtacttaaagttgatgctgaaggtcttccaactcttccgacaggcgactctgatcagttacgcgttggtgaatgggtagctgctattggatcaccattcggtttcgatcactctgttacagcaggtatcatttctgcgatcaatcgcactttacctcgtgatgcttacgtaccattcattcaaacggatgttgctatcaaccctggaaactctggtggtccgttattcaaccttgatggtgaggttgtaggcatcaacgcacagatctttacacgttctggtggtttcatgggtttgtcattcgcagttccgattaacgtagctatggatgtagcttctcaacttcgtgacgacggtcgtgttcgtcgcggttggttaggagttatgatccaacctgtttctcgtgatttagcagaaagcttcggaatggagtcagcatcaggagccttaatcgctgaccttgacccggaaggccctgctgcggaaggtggtgttcgcgcaggcgatatcatccttgaagttgacggtgaggaagttgaacgttcacgcaatcttccacgccttatcggtcgtgtttctccaggtaacgacgctgagttaacgttaatgcgtgacggtcaacgtgaacaagtaacagttacaatcggtgactggccagattctgacgaaccgcgtgcggatgcacgcgacgagcaatcagctcaagctcgtcttggcatcgctgtatctgagttagacgaaacagaacgcgaacaccttggagtagaagcaggcgtaatggttcgcgagatcgaaccggaaggcgctgcagctcgtgctggtattcgccgtggcgatgtaatcgtatctattgaccaccacacagtagagtctggatctcaattagttgagcttgtagaagaacttcctacagatcgcgctgttcctgttcgtctttaccgtgaaggtcgcagcttattcgttgctttacgtcttggacaggat

SEQ ID NO: 2 - protein, 477 aa, Halomonas sp
MRPITRSFPLWALLLALLLPWQAALARDLPDFANLVEQAAPGVVNISTTHTVERGQAFEGFGGQDIPDIFRHFFGDRFPGGPEGPMPGPGPGPSQERQSLGSGFIISEDGYILTSAHVVEGADEILVRLADRREMEAELVGADIQTDVAVLKVDAEGLPTLPTGDSDQLRVGEWVAAIGSPFGFDHSVTAGIISAINRTLPRDAYVPFIQTDVAINPGNSGGPLFNLDGEVVGINAQIFTRSGGFMGLSFAVPINVAMDVASQLRDDGRVRRGWLGVMIQPVSRDLAESFGMESASGALIADLDPEGPAAEGGVRAGDIILEVDGEEVERSRNLPRLIGRVSPGNDAELTLMRDGQREQVTVTIGDWPDSDEPRADARDEQSAQARLGIAVSELDETEREHLGVEAGVMVREIEPEGAAARAGIRRGDVIVSIDHHTVESGSQLVELVEELPTDRAVPVRLYREGRSLFVALRLGQD

SEQ ID NO: 3 - protein, 451 aa, Halomonas sp
RDLPDFANLVEQAAPGVVNISTTHTVERGQAFEGFGGQDIPDIFRHFFGDRFPGGPEGPMPGPGPGPSQERQSLGSGFIISEDGYILTSAHVVEGADEILVRLADRREMEAELVGADIQTDVAVLKVDAEGLPTLPTGDSDQLRVGEWVAAIGSPFGFDHSVTAGIISAINRTLPRDAYVPFIQTDVAINPGNSGGPLFNLDGEVVGINAQIFTRSGGFMGLSFAVPINVAMDVASQLRDDGRVRRGWLGVMIQPVSRDLAESFGMESASGALIADLDPEGPAAEGGVRAGDIILEVDGEEVERSRNLPRLIGRVSPGNDAELTLMRDGQREQVTVTIGDWPDSDEPRADARDEQSAQARLGIAVSELDETEREHLGVEAGVMVREIEPEGAAARAGIRRGDVIVSIDHHTVESGSQLVELVEELPTDRAVPVRLYREGRSLFVALRLGQD

SEQ ID NO: 4 - DNA, 1143 bp, Pseudomonas punonensis
  CDS (1)..(1140)
  sig_peptide (1)..(93)
  mat_peptide (94)..(1137)
atgctcaaggccctgcgttttcttggctggcccctgctggtcggcgtgctgctggcactgctgatcatgcagtattacccgcaatgggtcggcttgccgcgtcaggacgtgcagcttcgccaggcaccgatctacaaccgcacacaggaaggcccggtgtcctacgccaatgcggtgggtaccgcctcgccggcggtggccaacctgtacaccaccaagttcgtcagcaaaccgacccatccgctgttcgaggaccctcagttccgccgcttcttcggcgacaacctgcccaagcagcggcgcatggaatccagcctgggctctgcggtgatcatgagccctgaaggctatctgctgaccaacaaccacgtcaccaccggtgcggaccagatcgtcgtggccctgaaagatggccgcgaaaccctcgccagggtcattggcagcgacccggagaccgatctcgccgtgctcaagatcgacctggaaaacctgccgtcgatcaccctgggccgttccgacaatatcaagatcggcgatgtcaccctggccatcggcaaccccttcggcgtcggccagaccgtgaccatgggcatcatcagtgccaccgggcgcaaccagttgggcctcaacacctacgaagacttcatccagaccgatgcggcgatcaaccccggcaactcgggcggcgcgctggtcgacgcctacggcaacctggtcggcatcaacaccgcgatcttctccaaatccggcggctcgcaaggtatcggcttcgccattccggtcaagctggcgctggaggtgatgcaggccatcatcgagcacggccaggtgattcgcggctggctggggatcgaggtgcaaccgctgacgcccgaactggctgagtcctttggtctggacggccggcccgggatcgtcgttgccggtatctaccgggacggccccgcgcagaaagccggcctgcaacccggcgacctgatcctcaatatcgacggcgaacccgccggcgatggacgccgctcgatgaaccaggtggcgcgtgccaagcccggcgacaagatccgcatcgagatcatgcgcaacggcaagggcatggacctgaccgccgaaatcggcgtacgccctccggtaaacggcaactaa

SEQ ID NO: 5 - protein, 380 aa, Pseudomonas punonensis
MLKALRFLGWPLLVGVLLALLIMQYYPQWVGLPRQDVQLRQAPIYNRTQEGPVSYANAVGTASPAVANLYTTKFVSKPTHPLFEDPQFRRFFGDNLPKQRRMESSLGSAVIMSPEGYLLTNNHVTTGADQIVVALKDGRETLARVIGSDPETDLAVLKIDLENLPSITLGRSDNIKIGDVTLAIGNPFGVGQTVTMGIISATGRNQLGLNTYEDFIQTDAAINPGNSGGALVDAYGNLVGINTAIFSKSGGSQGIGFAIPVKLALEVMQAIIEHGQVIRGWLGIEVQPLTPELAESFGLDGRPGIVVAGIYRDGPAQKAGLQPGDLILNIDGEPAGDGRRSMNQVARAKPGDKIRIEIMRNGKGMDLTAEIGVRPPVNGN

SEQ ID NO: 6 - protein, 349 aa, Pseudomonas punonensis
LPRQDVQLRQAPIYNRTQEGPVSYANAVGTASPAVANLYTTKFVSKPTHPLFEDPQFRRFFGDNLPKQRRMESSLGSAVIMSPEGYLLTNNHVTTGADQIVVALKDGRETLARVIGSDPETDLAVLKIDLENLPSITLGRSDNIKIGDVTLAIGNPFGVGQTVTMGIISATGRNQLGLNTYEDFIQTDAAINPGNSGGALVDAYGNLVGINTAIFSKSGGSQGIGFAIPVKLALEVMQAIIEHGQVIRGWLGIEVQPLTPELAESFGLDGRPGIVVAGIYRDGPAQKAGLQPGDLILNIDGEPAGDGRRSMNQVARAKPGDKIRIEIMRNGKGMDLTAEIGVRPPVNGN

SEQ ID NO: 7 - DNA, 1131 bp, Cohnella sp
  CDS (1)..(1128)
  sig_peptide (1)..(90)
  mat_peptide (91)..(1125)
atggcaaaatttcgaggacttcggacccgacttcccgcggctctggcggccgcttcgctgctgtggttcgccgcgtcggaagccgaggcggtcgcgccggcttcgctctcgttggacgcgaaggtgatcgacggcgaagtctacgtcaaggcttcttcgctcgtggccgccgcgggcggcgccggctcttacaactccgcgacgaagaagtacgactatacgcccgcgaacgccgtgccggacgtcgtgaagaaggtcgcgccttcggtcgtcgcgatcatcggcaagccgcaggacggctcgtccgacaaccgcttctcgctcgcgcacggcaccggcgtcgtcgtgcggcaggacggcctcatcctgacgaacgcccacgtcgtcaatgacatggaccgcatcgtcgtcgtcacgagcgacggcaaggagtacgacggcacgaagaagcagatcgacgagacgagcgatctcgcgctcgtgaagatcaacgccgcgaacctgaagccggttacgttcgcgccggcgccgctgaacgttcaggtcggcgagacggtcgtggccatcggcacgcccgtgtcgttcgcgcttcggaacacggcgacggtcggggtgatcagcgggctgaaccggtccgtatcttcctattacaacctgctgcagacggacgcggccatcaatccgggcaacagcggcggtccgctcgtcaatctgaatggacaagtcgtcggcatcaactcgatgaagttcgtcgacgtcgacatcgacagcatgggcttctcgattccggcggataccgtgcaatacgtcctgaatcaattcaacaagaacggcaaagtcgtccgtgcgacgctcggcctcgagctggaggagagctggtcggcggtggtcggcttgcccacgcaggatccgatgaccgtaacggcggtcacggcggccgcggcgaagaacgccggcatcgaggtcggggacaagctgtactcgatcgcgggcaagcaggtctccacggtggtggagatgaacgagctgctcaaggcgtacacgccgggccaaacggtatcggtcacgatgctgtcggacggcgacctggtcacgcgcaagatcgtgctgtcgacaagctcggaccattaa

SEQ ID NO: 8 - protein, 376 aa, Cohnella sp
MAKFRGLRTRLPAALAAASLLWFAASEAEAVAPASLSLDAKVIDGEVYVKASSLVAAAGGAGSYNSATKKYDYTPANAVPDVVKKVAPSVVAIIGKPQDGSSDNRFSLAHGTGVVVRQDGLILTNAHVVNDMDRIVVVTSDGKEYDGTKKQIDETSDLALVKINAANLKPVTFAPAPLNVQVGETVVAIGTPVSFALRNTATVGVISGLNRSVSSYYNLLQTDAAINPGNSGGPLVNLNGQVVGINSMKFVDVDIDSMGFSIPADTVQYVLNQFNKNGKVVRATLGLELEESWSAVVGLPTQDPMTVTAVTAAAAKNAGIEVGDKLYSIAGKQVSTVVEMNELLKAYTPGQTVSVTMLSDGDLVTRKIVLSTSSDH

SEQ ID NO: 9 - protein, 346 aa, Cohnella sp
VAPASLSLDAKVIDGEVYVKASSLVAAAGGAGSYNSATKKYDYTPANAVPDVVKKVAPSVVAIIGKPQDGSSDNRFSLAHGTGVVVRQDGLILTNAHVVNDMDRIVVVTSDGKEYDGTKKQIDETSDLALVKINAANLKPVTFAPAPLNVQVGETVVAIGTPVSFALRNTATVGVISGLNRSVSSYYNLLQTDAAINPGNSGGPLVNLNGQVVGINSMKFVDVDIDSMGFSIPADTVQYVLNQFNKNGKVVRATLGLELEESWSAVVGLPTQDPMTVTAVTAAAAKNAGIEVGDKLYSIAGKQVSTVVEMNELLKAYTPGQTVSVTMLSDGDLVTRKIVLSTSSDH

SEQ ID NO: 10 - DNA, 1188 bp, Bacillus sp
  CDS (1)..(1185)
  sig_peptide (1)..(123)
  mat_peptide (124)..(1182)
atgggattttatgatgatgaattcgatacaaagccgaagcgttcgcgagggaacgtttcctcttgggtggcagtcgcattggtatccgcgttgattggttcgggtacaacgatcgctctcgttcctacacttatcaaatcaaatgtcattcaaacaaatggaaatggcatccaatcaacatctgcttccggcaatgtgtccggcttgcaacaaacgacgagcgtgagcgtcaacgacggtgtggtgcaagcggtcaacaaagtaaaaccttccatcgttggtgtgtcgaacatgcaggcgcaacaaaacgcatttggcagcaaatcgggttcgctgcaggaagcgggcaccggttcggggatcttgttcgacgatcaaggacatatcgttaccaataaccatgtcgtggaaggggccagtgaagtcgatattaccttgctggacggcacgcatgtaaaagcgcaagtcgtcgggacagacccgatcaccgatttggctgtgctcaaagtggatccgagcaaaatcaaaggtctgaaaccggctacgtttggcaacagtgacacgctgaatatcggggagccggcgattgcgatcgggaacccgttgggcatgagtttcgcgcagaccgtaacggtcggcgtcatctcggcgaccaaccgcgatatgcctgtgcaagacgaacagacgggtcagacgctgtacagccaaacagtgttgcaaacggacgccgcgatcaaccccggcaactccggcggtgcgctttgcaacatcaacggccaagtcatcggcattaactccgcgaagatcgcaacgtccggcgtggaggggatcggatttgcgattccgatcaacgaagcgcgtccgatcatccaacagttgcttgaaaatgggaaagttgttcgtccgcagttgggcgttgccggttacaacctggcagaagtgcctgcttattaccagccgaatgtgccgaccaatacagggatcttggtagaacaagcacaaggcggtgcggcacaggccggactgaccaagggtgacgtgatcgtaaaagtcgacggtcaagaggttgatgaccaagcttctctgcgaaaagtgttgttcacccataagccgggtgacactgtgaaagtggaattctaccgtggcaatacggagaaaacggtcgatgtaaaattggcttcttcttaa

SEQ ID NO: 11 - protein, 395 aa, Bacillus sp
MGFYDDEFDTKPKRSRGNVSSWVAVALVSALIGSGTTIALVPTLIKSNVIQTNGNGIQSTSASGNVSGLQQTTSVSVNDGVVQAVNKVKPSIVGVSNMQAQQNAFGSKSGSLQEAGTGSGILFDDQGHIVTNNHVVEGASEVDITLLDGTHVKAQVVGTDPITDLAVLKVDPSKIKGLKPATFGNSDTLNIGEPAIAIGNPLGMSFAQTVTVGVISATNRDMPVQDEQTGQTLYSQTVLQTDAAINPGNSGGALCNINGQVIGINSAKIATSGVEGIGFAIPINEARPIIQQLLENGKVVRPQLGVAGYNLAEVPAYYQPNVPTNTGILVEQAQGGAAQAGLTKGDVIVKVDGQEVDDQASLRKVLFTHKPGDTVKVEFYRGNTEKTVDVKLASS

SEQ ID NO: 12 - protein, 354 aa, Bacillus sp
PTLIKSNVIQTNGNGIQSTSASGNVSGLQQTTSVSVNDGVVQAVNKVKPSIVGVSNMQAQQNAFGSKSGSLQEAGTGSGILFDDQGHIVTNNHVVEGASEVDITLLDGTHVKAQVVGTDPITDLAVLKVDPSKIKGLKPATFGNSDTLNIGEPAIAIGNPLGMSFAQTVTVGVISATNRDMPVQDEQTGQTLYSQTVLQTDAAINPGNSGGALCNINGQVIGINSAKIATSGVEGIGFAIPINEARPIIQQLLENGKVVRPQLGVAGYNLAEVPAYYQPNVPTNTGILVEQAQGGAAQAGLTKGDVIVKVDGQEVDDQASLRKVLFTHKPGDTVKVEFYRGNTEKTVDVKLASS

SEQ ID NO: 13 - DNA, 1329 bp, Psychrobacter arcticus
  CDS (1)..(1326)
  sig_peptide (1)..(117)
  mat_peptide (118)..(1323)
atgtcaagcagcccaaacgcaaacaacaaagctggtcttcttcgctggcttccatggatccttcttcttgtagttacggctgcgtttatctggcttttcgcttcaatgaaaactgcttcagaagcaaaatgggaaccgccacgcactactcctgcagagcaacaagcttctgaaccagtttctgacgcttctacgcctatcgcatcttaccataacggagttgctcgcgcatctcaatctgttgttaacatctatactacacagacgatggcggagcatccttacatgaacgacccagttcttcgtcgcttctttgaatatcatggcggtccaagcgaggagcagggcaacacaaaccttggttcaggagttatcgtttctgaggacggctatatcgttactaacgcacacgttatcgagaaagctgacgagatcacagtagctttcaacgatggtcgcaagtctcgtgcgaaacttatcggcactgaccctgacagcgaccttgctgttatcaaagtagacatgactggtcttacgccacttggattccgcgagaaaccaacgcttgttggcgacgtagcacttgctatcggaaacccattcggtgttggccagacgatcactcaaggcatcatctcagcaacaggtcgcactggccttggcgtaaacaaattcgaggacttcatccagacggacgcagcgatcaacccaggaaactcaggtggagctcttgtagacgctcgcggtgagcttatcggtatcaacactgcaatctacagccgcagcggtggttctatgggaatcggttttgctatcccgacggcgatcgtagagcaagtaatgaacgcacttatcaaagacggcaaggtatctcgcggttggcttggaatcgagatccaatctcaacttcgcgacccaacacaacttgagacatcaacaggagtagaggttcttgacgtagtaccaaaaagccctgctgcaaaatctggacttaaagttggagacatcgtacttacaatcgacggagttgagatgacggacgcgaacacgcttatccaatatgtagcacgcaaagcgccaaacacaacacttcaagctcagatccttcgccaaggtaagaacgcgcaagttaagatccttcttgaagaacgtccggttgaggagcctgttactgttccggtagtaatccaagacgagatgcttggtggtgcgcagccaccatcacttcaagcacctgagcaagacactccaatgatgtctgaagctgagcgcgaccgtatgcgcgaggagcttcttcttcttttcgagcgcgacggagctccactttaa

SEQ ID NO: 14 - protein, 442 aa, Psychrobacter arcticus
MSSSPNANNKAGLLRWLPWILLLVVTAAFIWLFASMKTASEAKWEPPRTTPAEQQASEPVSDASTPIASYHNGVARASQSVVNIYTTQTMAEHPYMNDPVLRRFFEYHGGPSEEQGNTNLGSGVIVSEDGYIVTNAHVIEKADEITVAFNDGRKSRAKLIGTDPDSDLAVIKVDMTGLTPLGFREKPTLVGDVALAIGNPFGVGQTITQGIISATGRTGLGVNKFEDFIQTDAAINPGNSGGALVDARGELIGINTAIYSRSGGSMGIGFAIPTAIVEQVMNALIKDGKVSRGWLGIEIQSQLRDPTQLETSTGVEVLDVVPKSPAAKSGLKVGDIVLTIDGVEMTDANTLIQYVARKAPNTTLQAQILRQGKNAQVKILLEERPVEEPVTVPVVIQDEMLGGAQPPSLQAPEQDTPMMSEAERDRMREELLLLFERDGAPL

SEQ ID NO: 15 - protein, 403 aa, Psychrobacter arcticus
SEAKWEPPRTTPAEQQASEPVSDASTPIASYHNGVARASQSVVNIYTTQTMAEHPYMNDPVLRRFFEYHGGPSEEQGNTNLGSGVIVSEDGYIVTNAHVIEKADEITVAFNDGRKSRAKLIGTDPDSDLAVIKVDMTGLTPLGFREKPTLVGDVALAIGNPFGVGQTITQGIISATGRTGLGVNKFEDFIQTDAAINPGNSGGALVDARGELIGINTAIYSRSGGSMGIGFAIPTAIVEQVMNALIKDGKVSRGWLGIEIQSQLRDPTQLETSTGVEVLDVVPKSPAAKSGLKVGDIVLTIDGVEMTDANTLIQYVARKAPNTTLQAQILRQGKNAQVKILLEERPVEEPVTVPVVIQDEMLGGAQPPSLQAPEQDTPMMSEAERDRMREELLLLFERDGAPL

SEQ ID NO: 16 - DNA, 1200 bp, Paenibacillus sp
  CDS (1)..(1197)
  sig_peptide (1)..(87)
  mat_peptide (88)..(1194)
atgcaaaagcgcaaattcaactcgatagcaagaggtatcattcccgcttttttatccggcgctttggtgatcgggggcttgatgttcgcctcggaccgcatgaatctgttcaccggaggacagcttattgacggttcggcttatactgacgacagccgtgcttcgggcggctccgaatcggcacaggtgcagaccgtttcggctgcgacggcatccaccggcaaatcaatcgccgaaatcgccgagttatcaagcccggcggttgtgaaaattacgtcgatgacgaaggcttcttccctgatgcagggacgcgatttttaccgcaaccaggggcagtcggacgagctgctggagagcggcagcggcacgggatttatttttgacaaaagcggctatattttgacaaacgagcatgtgattcaaggcgccgacaaagtggaagtgtccgtgcaaggatacgtcgacacgttcacggcaaccgtgctcggcagcagctacgatctcgatctggccgtgttgaaaatcgatgcaggcgacgagttgcccgccttgacgttgggcgattccgatgcgatgcagatcggggagtgggtcgtcgccatcggcaatccgtacgatctggattatacggttacgaccggggtcgtcagcgcaagagaacgtacgattaccattgaagattcggaaggcgcgcgcaactaccagcatctgttccagaccgatacggcgatcaatccgggcaattccggcggcccgctgctcaatttgtccggtgacgtgatcggtattaataccgcggtcagcagcggcgctcaagggatcgggtttgcgattccttccaacacggtgaaggcggtgctcgagaagctgatccataatgaaagcattccaatcgcctatatgggcgtcagcctgcaggatgtgaacgaaacgatccagaacgggctgaacttaaccggcgcaaaaggcgcgctcgtcggtcatgtgcaagccggctcgccggccgagaagtccgggctgcggcagtatgatgtcattacggccgccaatggcgaggcaattccagacgcttccgccatgacccataaaattctccaatcgcaagtgggcgacgaggtgaagctgacggtcatccgcgacggacggacaatggaattggcggttgtattgggcgacaaaagcggaggtatgcaaggttga

SEQ ID NO: 17 - protein, 399 aa, Paenibacillus sp
MQKRKFNSIARGIIPAFLSGALVIGGLMFASDRMNLFTGGQLIDGSAYTDDSRASGGSESAQVQTVSAATASTGKSIAEIAELSSPAVVKITSMTKASSLMQGRDFYRNQGQSDELLESGSGTGFIFDKSGYILTNEHVIQGADKVEVSVQGYVDTFTATVLGSSYDLDLAVLKIDAGDELPALTLGDSDAMQIGEWVVAIGNPYDLDYTVTTGVVSARERTITIEDSEGARNYQHLFQTDTAINPGNSGGPLLNLSGDVIGINTAVSSGAQGIGFAIPSNTVKAVLEKLIHNESIPIAYMGVSLQDVNETIQNGLNLTGAKGALVGHVQAGSPAEKSGLRQYDVITAANGEAIPDASAMTHKILQSQVGDEVKLTVIRDGRTMELAVVLGDKSGGMQG

SEQ ID NO: 18 - protein, 370 aa, Paenibacillus sp
ASDRMNLFTGGQLIDGSAYTDDSRASGGSESAQVQTVSAATASTGKSIAEIAELSSPAVVKITSMTKASSLMQGRDFYRNQGQSDELLESGSGTGFIFDKSGYILTNEHVIQGADKVEVSVQGYVDTFTATVLGSSYDLDLAVLKIDAGDELPALTLGDSDAMQIGEWVVAIGNPYDLDYTVTTGVVSARERTITIEDSEGARNYQHLFQTDTAINPGNSGGPLLNLSGDVIGINTAVSSGAQGIGFAIPSNTVKAVLEKLIHNESIPIAYMGVSLQDVNETIQNGLNLTGAKGALVGHVQAGSPAEKSGLRQYDVITAANGEAIPDASAMTHKILQSQVGDEVKLTVIRDGRTMELAVVLGDKSGGMQG

SEQ ID NO: 19 - DNA, 1215 bp, Halomonas sp
  CDS (1)..(1215)
  sig_peptide (1)..(90)
  mat_peptide (91)..(1212)
atgcgtcgccctttacttgcttacgctttaccagttcttatcggagttcttttagcaatcgtacttcttaacacttttccacaattacttggtccttctcaaccagaacctcgtgcccctacatctgaagtagaagctccttctgaacttcaacaaccagcatcacgtgccacacctgaagtacgtcaagcagctcctttatcacgtatgcaaggtcctgttagctattctgaagctgtagaaaaggctgctccagctgttgttaacatttactcttctcgtgttgtagcgcttgaagaacatccaatgatgtctgacccgttctttcgtcaattctttggcgaggatttaccatctcaacaacgctttctttctagccttggctcaggtgtaattgtaagcgaagacggttatgtacttacgaaccaccatgttatcgatggagctgatgaaatccaagttgctcttcgcgatggacgtgaaacacttgcggaagtaatcggcacagatcctgaatctgacttagcagttcttcgtatcgatcttgatgaccttccggttattgttctttatgatactgaggatgttgctgttggtgatattgcgatggctatcggtaatcctttcggcgttggtcaaacagtaactatgggcattatctctgcaacaggtcgtactcacttaggtctttcagcttacgaggacttcattcaaactgacgcagcaatcaaccctggtaattctggtggagcattagtaaacgctgaaggtgctcttgttggcattaacactgcgatcttttcacgctcaggtggctctcaaggagttggttttgcgattccaactcgccttgctcactctatccttgacgatcttgtaactcaaggccgtgttatccgtggttggcttggcattgatgctcaggagattacgccagaacttgcttcttctttcggtttgcaagctccacgtggagtagtaatctctggtgttgtaccagaaggtcctgcagcacgtgctggtcttcgtccaggtgacgtacttcttgaagttgatggtcgtccaatccttgacgcacgcgtagcaatgtctgacattgcagcaatccagccaggtgctacacttccgcttacagtagttcgtggcggagaacgtcttggcatcgagcttgaagttggtgaacgtccgcctccgcaacttcgtcaaccagaacgc

SEQ ID NO: 20 - protein, 405 aa, Halomonas sp
MRRPLLAYALPVLIGVLLAIVLLNTFPQLLGPSQPEPRAPTSEVEAPSELQQPASRATPEVRQAAPLSRMQGPVSYSEAVEKAAPAVVNIYSSRVVALEEHPMMSDPFFRQFFGEDLPSQQRFLSSLGSGVIVSEDGYVLTNHHVIDGADEIQVALRDGRETLAEVIGTDPESDLAVLRIDLDDLPVIVLYDTEDVAVGDIAMAIGNPFGVGQTVTMGIISATGRTHLGLSAYEDFIQTDAAINPGNSGGALVNAEGALVGINTAIFSRSGGSQGVGFAIPTRLAHSILDDLVTQGRVIRGWLGIDAQEITPELASSFGLQAPRGVVISGVVPEGPAARAGLRPGDVLLEVDGRPILDARVAMSDIAAIQPGATLPLTVVRGGERLGIELEVGERPPPQLRQPER

SEQ ID NO: 21 - protein, 375 aa, Halomonas sp
GPSQPEPRAPTSEVEAPSELQQPASRATPEVRQAAPLSRMQGPVSYSEAVEKAAPAVVNIYSSRVVALEEHPMMSDPFFRQFFGEDLPSQQRFLSSLGSGVIVSEDGYVLTNHHVIDGADEIQVALRDGRETLAEVIGTDPESDLAVLRIDLDDLPVIVLYDTEDVAVGDIAMAIGNPFGVGQTVTMGIISATGRTHLGLSAYEDFIQTDAAINPGNSGGALVNAEGALVGINTAIFSRSGGSQGVGFAIPTRLAHSILDDLVTQGRVIRGWLGIDAQEITPELASSFGLQAPRGVVISGVVPEGPAARAGLRPGDVLLEVDGRPILDARVAMSDIAAIQPGATLPLTVVRGGERLGIELEVGERPPPQLRQPER

SEQ ID NO: 22 - protein, 27 aa, Artificial
  Signal
MKKPLGKIVASTALLISVAFSSSIASA

SEQ ID NO: 23 - protein, 6 aa, Artificial
  His-tag
HHHHHH

(19) United States
(12) Patent Application Publication
(10) Pub. No.: US 2022/0411773 A1
(43) Pub. Date: Dec. 29, 2022

(54) POLYPEPTIDES HAVING PROTEOLYTIC ACTIVITY AND USE THEREOF

(71) Applicant: Novozymes A/S, Bagsvaerd (DK)

(72) Inventors: Klaus Gori, Dyssegaard (DK); Jesper Salomon, Holte (DK); Lorena Gonzalez Palmen, Akarp (SE); Marc Dominique Morant, Frederiksberg (DK)

(73) Assignee: Novozymes A/S, Bagsvaerd (DK)

(21) Appl. No.: 17/778,452

(22) PCT Filed: Dec. 18, 2020
(86) PCT No.: PCT/EP2020/087222

§ 371 Date: May 20, 2022

(30) Foreign Application Priority Data
Dec. 20, 2019 (EP) 19218813.4

(51) Int. Cl. C12N 9/52; C12N 5/00; C11D 3/386

(52) U.S. Cl. CPC C12N 9/52; C12N 5/0018; C11D 3/38609; C11D 3/38672; C12N 2501/734

(57) ABSTRACT

The present invention relates to isolated polypeptides having proteolytic activity and polynucleotides encoding the polypeptides. The invention also relates to nucleic acid constructs, vectors and host cells comprising the polynucleotides, compositions comprising the polypeptides, and methods of producing and using the polypeptides.

### REFERENCE TO A SEQUENCE LISTING

This application contains a Sequence Listing in computer readable form, which is incorporated herein by reference.

### FIELD OF THE INVENTION

The present invention relates to polypeptides having proteolytic activity and polynucleotides encoding the polypeptides, nucleic acid constructs, vectors, and host cells comprising the polynucleotides, as well as methods of producing and using the polypeptides.

The object of the present invention is to provide novel enzymes and enzyme compositions suitable for cleaning applications, such as household cleaning compositions for laundry or dishwashing. Compositions comprising the polypeptides of the invention are for example suitable for removing biofilm from textiles.

### SUMMARY OF THE INVENTION

A first aspect of the invention relates to an isolated or purified polypeptide having proteolytic activity, selected from the group consisting of:

(a)
i. a polypeptide having at least 96%, at least 97%, at least 98%, at least 99% or at least 100% sequence identity to SEQ ID NO: 21;
ii. a polypeptide having at least 92%, at least 93%, at least 94%, at least 95%, at least 96%, at least 97%, at least 98%, at least 99% or at least 100% sequence identity to SEQ ID NO: 3,
iii. a polypeptide having at least 94%, at least 95%, at least 96%, at least 97%, at least 98%, at least 99% or at least 100% sequence identity to sequence identity to SEQ ID NO: 6,
iv. a polypeptide having at least 81%, at least 85%, at least 90%, at least 95%, at least 96%, at least 97%, at least 98%, at least 99% or at least 100% sequence identity to sequence identity to SEQ ID NO: 9,
v. a polypeptide having at least 60%, at least 65%, at least 70%, at least 75, at least 80%, at least 85%, at least 90%, at least 95%, at least 96%, at least 97%, at least 98%, at least 99% or at least 100% sequence identity to SEQ ID NO: 12, or
vi. a polypeptide having at least 55%, at least 60%, at least 65%, at least 70%, at least 75, at least 80%, at least 85%, at least 90%, at least 95%, at least 96%, at least 97%, at least 98%, at least 99% or at least 100% sequence identity to SEQ ID NO: 18,

(b) a polypeptide encoded by a polynucleotide that hybridizes under high stringency conditions with the full-length complement of the mature polypeptide coding sequence of SEQ ID NO: 19, 1, 4, 7, 10, 13 or 16;

(c) a polypeptide derived from a polypeptide of SEQ ID NO: 21, 3, 6, 9, 12, 15 or 18 by substitution, deletion or addition of one or several amino acids in the polypeptide of SEQ ID NO: 21, 3, 6, 9, 12, 15 or 18; and

(d) a fragment of the polypeptide of (a), (b) or (c) that has proteolytic activity.

A second aspect of the invention relates to a granule, which comprises:

(a) a core comprising the polypeptide of the invention, and, optionally

(b) a coating consisting of one or more layer(s) surrounding the core.

A third aspect relates to a granule, which comprises:

(a) a core, and

(b) a coating consisting of one or more layer(s) surrounding the core, wherein the coating comprises the polypeptide according to the invention.

A fourth aspect of the invention relates to a composition comprising the polypeptide or the granule of the invention.

The invention further relates to a whole broth formulation or cell culture composition comprising a polypeptide of the invention, and a nucleic acid construct or expression vector comprising a polynucleotide of the invention, wherein the polynucleotide is operably linked to one or more control sequences that direct the production of the polypeptide in an expression host. The invention also relates to a recombinant host cell comprising the polynucleotide of the invention operably linked to one or more control sequences that direct the production of the polypeptide. The invention further relates to a method of producing a polypeptide having proteolytic activity, comprising cultivating the recombinant host cell under conditions conducive for production of the polypeptide, and optionally recovering a polypeptide of the invention. The invention also relates to use of the polypeptide and compositions comprising the polypeptide for cleaning, as well as methods of cleaning using the polypeptides and compositions.

### Definitions

Polypeptide having proteolytic activity: The term “polypeptide having proteolytic activity” means a protease (also called peptidases or proteinases), which is an enzyme that catalyzes the proteolysis i.e. the breakdown of proteins into smaller polypeptides or single amino acids. Proteases may be classified into seven groups: Serine proteases—using a serine alcohol, Cysteine proteases—using a cysteine thiol, Threonine proteases—using a threonine secondary alcohol, Aspartic proteases—using an aspartate carboxylic acid, Glutamic proteases—using a glutamate carboxylic acid, Metalloproteases—using a metal, usually zinc, Asparagine peptide lyases—using an asparagine to perform an elimination reaction (not requiring water) activity.

A protease of the invention may be classified in any of the seven groups. Preferably the protease is a Serine protease, Trypsin protease or any homologues hereof. The most widely used proteases in household cleaning compositions are the serine proteases. These have shown to be stable in detergents and at elevated temperatures. The most relevant of these proteases are alkaline. Proteolytic activity can be determined as described in Assay I herein. In one aspect, the polypeptides of the present invention have at least 20%, e.g., at least 40%, at least 50%, at least 60%, at least 70%, at least 80%, at least 90%, at least 95%, or at least 100% of the proteolytic activity of the mature polypeptide of SEQ ID NO: 2, 5, 8, 11, 14, 17 or 20.

Catalytic domain: The term “catalytic domain” means the region of an enzyme containing the catalytic machinery of the enzyme.

cDNA: The term “cDNA” means a DNA molecule that can be prepared by reverse transcription from a mature, spliced mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cleaning component: The cleaning component, which may also be referred to as a detergent adjunct ingredient, is different from the polypeptide having proteolytic activity. The precise nature of these additional cleaning components e.g. adjunct components, and levels of incorporation thereof, will depend on the physical form of the composition and the nature of the operation for which it is to be used. Suitable cleaning components e.g. adjunct materials include, but are not limited to the components described below such as surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric agents, clay soil removal/anti-redeposition agents, brighteners, suds suppressors, dyes, perfumes, structure elasticizing agents, fabric softeners, carriers, hydrotropes, builders and co-builders, fabric hueing agents, anti-foaming agents, dispersants, processing aids, and/or pigments.

Cleaning composition: The term “cleaning composition” or “detergent composition” refers to compositions that find use in the removal of undesired compounds from items to be cleaned, such as textiles and hard surfaces. The cleaning composition may be used to e.g. clean textiles or hard surfaces (e.g. dishes) for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). In addition to containing the polypeptide having proteolytic activity, the cleaning composition may contain e.g. detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Coding sequence: The term “coding sequence” means a polynucleotide which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a polypeptide of the present invention. Each control sequence may be native (i.e., from the same gene) or heterologous (i.e., from a different gene) to the polynucleotide encoding the polypeptide or native or heterologous to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” means any step involved in the production of a polypeptide including, but not limited to, transcription, post-transcriptional modification, translation, post-translational modification and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more amino acids absent from the amino and/or carboxyl terminus of a mature polypeptide or domain; wherein the fragment has protease activity.

In some embodiments, a fragment contains at least 380 amino acid residues (e.g., amino acids 70 to 450 of SEQ ID NO: 3), at least 330 amino acid residues (e.g., amino acids 70 to 400 of SEQ ID NO: 3), at least 205 amino acid residues (e.g., amino acids 240 to 445 of SEQ ID NO: 3), at least 195 amino acid residues (e.g., amino acids 240 to 435 of SEQ ID NO: 3), at least 95 amino acid residues (e.g., amino acids 245 to 340 of SEQ ID NO: 3), or at least 65 amino acid residues (e.g., amino acids 350 to 415 of SEQ ID NO: 3). In some embodiments, a fragment contains at least 270 amino acid residues (e.g., amino acids 70 to 340 of SEQ ID NO: 6), at least 180 amino acid residues (e.g., amino acids 70 to 250 of SEQ ID NO: 6), at least 265 amino acid residues (e.g., amino acids 75 to 340 of SEQ ID NO: 6), at least 95 amino acid residues (e.g., amino acids 245 to 340 of SEQ ID NO: 6), at least 90 amino acid residues (e.g., amino acids 250 to 340 of SEQ ID NO: 6), or at least 65 amino acid residues (e.g., amino acids 275 to 340 of SEQ ID NO: 6).

In some embodiments, a fragment contains at least 275 amino acid residues (e.g., amino acids 75 to 350 of SEQ ID NO: 9), at least 155 amino acid residues (e.g., amino acids 80 to 235 of SEQ ID NO: 9), at least 152 amino acid residues (e.g., amino acids 81 to 233 of SEQ ID NO: 9), at least 126 amino acid residues (e.g., amino acids 107 to 233 of SEQ ID NO: 9) or at least 70 amino acid residues (e.g., amino acids 270 to 340 of SEQ ID NO: 9).

In some embodiments, a fragment contains at least 180 amino acid residues (e.g., amino acids 70 to 250 of SEQ ID NO: 12), at least 170 amino acid residues (e.g., amino acids 75 to 245 of SEQ ID NO: 12), at least 164 amino acid residues (e.g., amino acids 77 to 241 of SEQ ID NO: 12), at least 285 amino acid residues (e.g., amino acids 70 to 355 of SEQ ID NO: 12) or at least 70 amino acid residues (e.g., amino acids 283 to 351 of SEQ ID NO: 12).

In some embodiments, a fragment contains at least 140 amino acid residues (e.g., amino acids 75 to 215 of SEQ ID NO: 15), at least 265 amino acid residues (e.g., amino acids 80 to 345 of SEQ ID NO: 15), at least 175 amino acid residues (e.g., amino acids 80 to 255 of SEQ ID NO:

15), at least 80 amino acid residues (e.g., amino acids 245 to 325 of SEQ ID NO: 15), at least 85 amino acid residues (e.g., amino acids 260 to 345 of SEQ ID NO: 15) or at least 50 amino acid residues (e.g., amino acids 275 to 325 of SEQ ID NO: 15).

In some embodiments, a fragment contains at least 145 amino acid residues (e.g., amino acids 90 to 235 of SEQ ID NO: 18), at least 160 amino acid residues (e.g., amino acids 90 to 250 of SEQ ID NO: 18), at least 175 amino acid residues (e.g., amino acids 93 to 276 of SEQ ID NO: 18), at least 175 amino acid residues (e.g., amino acids 90 to 265 of SEQ ID NO: 18), at least 85 amino acid residues (e.g., amino acids 270 to 360 of SEQ ID NO: 18), at least 80 amino acid residues (e.g., amino acids 270 to 350 of SEQ ID NO: 18) or at least 55 amino acid residues (e.g., amino acids 295 to 350 of SEQ ID NO: 18).

In some embodiments, a fragment contains at least 180 amino acid residues (e.g., amino acids 90 to 270 of SEQ ID NO: 21), at least 165 amino acid residues (e.g., amino acids 90 to 255 of SEQ ID NO: 21), at least 265 amino acid residues (e.g., amino acids 95 to 360 of SEQ ID NO: 21), at least 85 amino acid residues (e.g., amino acids 265 to 350 of SEQ ID NO: 21), at least 85 amino acid residues (e.g., amino acids 270 to 360 of SEQ ID NO: 21), at least 80 amino acid residues (e.g., amino acids 270 to 350 of SEQ ID NO: 21) or at least 60 amino acid residues (e.g., amino acids 270 to 330 of SEQ ID NO: 21).

Fusion polypeptide: The term “fusion polypeptide” means a polypeptide in which a polypeptide is fused at the N-terminus or the C-terminus of a polypeptide of the present invention. A fusion polypeptide is produced by fusing a polynucleotide encoding another polypeptide to a polynucleotide of the present invention. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Hard surface cleaning: The term “hard surface cleaning” is defined herein as cleaning of hard surfaces wherein hard surfaces may include floors, tables, walls, roofs etc. as well as surfaces of hard objects such as cars (car wash) and dishes (dishwashing). Dishwashing includes but is not limited to cleaning of plates, cups, glasses, bowls, cutlery such as spoons, knives, forks, serving utensils, ceramics, plastics, metals, china, glass and acrylics.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, or domain of a polypeptide or nucleic acid is not naturally associated with the polypeptide or nucleic acid, e.g., the control sequence is from a gene other than the gene encoding the mature polypeptide of SEQ ID NO: 2.

Host cell: The term “host cell” means any microbial or plant cell into which a nucleic acid construct or expression vector comprising a polynucleotide of the present invention has been introduced. Methods for introduction include but are not limited to protoplast fusion, transfection, transformation, electroporation, conjugation, and transduction. In some embodiments, the host cell is an isolated recombinant host cell that is partially or completely separated from at least one other component with, including but not limited to, proteins, nucleic acids, cells, etc.

Hybrid polypeptide: The term “hybrid polypeptide” means a polypeptide comprising domains from two or more polypeptides, e.g., a binding module from one polypeptide and a catalytic domain from another polypeptide. The domains may be fused at the N-terminus or the C-terminus.

Hybridization: The term “hybridization” means the pairing of substantially complementary strands of nucleic acids, using standard Southern blotting procedures. Hybridization may be performed under medium, medium-high, high or very high stringency conditions. Medium stringency conditions means prehybridization and hybridization at 42° C. in 5× SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 55° C. Medium-high stringency conditions means prehybridization and hybridization at 42° C. in 5× SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 60° C. High stringency conditions means prehybridization and hybridization at 42° C. in 5× SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 65° C. Very high stringency conditions means prehybridization and hybridization at 42° C. in 5× SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 70° C.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture broth containing the secreted polypeptide.

Laundering: The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

Malodor: By the term “malodor” is meant an odor which is not desired on clean items. One example of malodor is an unpleasant smell caused by compounds which may be produced by microorganisms. Another example is unpleasant smells caused by sweat or body odor adhered to an item which has been in contact with a human or animal. Another example of malodor can be the odor from spices, which stick to items such as curry or other spices which smell strongly.

Mature polypeptide: The term “mature polypeptide” means a polypeptide in its mature form following N-terminal processing (e.g., removal of signal peptide). In one aspect, the mature polypeptide is SEQ ID NO: 3. In one aspect, the mature polypeptide is SEQ ID NO: 6. In one aspect, the mature polypeptide is SEQ ID NO: 9. In one aspect, the mature polypeptide is SEQ ID NO: 15. In one aspect, the mature polypeptide is SEQ ID NO: 18. In one aspect, the mature polypeptide is SEQ ID NO: 21.

Mature polypeptide coding sequence: The term “mature polypeptide coding sequence” means a polynucleotide that encodes a mature polypeptide having proteolytic activity. In one aspect, the mature polypeptide coding sequence is nucleotides 79 to 1428 of SEQ ID NO: 1. In one aspect, the mature polypeptide coding sequence is nucleotides 94 to 1137 of SEQ ID NO: 4. In one aspect, the mature polypeptide coding sequence is nucleotides 91 to 1125 of SEQ ID NO:

7. In one aspect, the mature polypeptide coding sequence is nucleotides 124 to 1182 of SEQ ID NO: 10. In one aspect, the mature polypeptide coding sequence is nucleotides 118 to 1323 of SEQ ID NO: 13. In one aspect, the mature polypeptide coding sequence is nucleotides 88 to 1194 of SEQ ID NO: 16. In one aspect, the mature polypeptide coding sequence is nucleotides 91 to 1212 of SEQ ID NO: 19.

Native: The term “native” means a nucleic acid or polypeptide naturally occurring in a host cell.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Purified: The term “purified” means a nucleic acid or polypeptide that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used, it is intended to include the broader term textiles as well.

Variant: The term “variant” means a polypeptide having proteolytic activity comprising a man-made mutation, i.e., a substitution, insertion, and/or deletion (e.g., truncation), at one or more positions. A substitution means replacement of the amino acid occupying a position with a different amino acid; a deletion means removal of the amino acid occupying a position; and an insertion means adding an amino acid adjacent to and immediately following the amino acid occupying a position

Wash performance: The term “wash performance” is used as an enzyme's ability to remove stains present on the object to be cleaned during e.g. wash or hard surface cleaning.

Wild-type: The term “wild-type” in reference to an amino acid sequence or nucleic acid sequence means that the amino acid sequence or nucleic acid sequence is a native or naturally occurring sequence. As used herein, the term “naturally occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### DETAILED DESCRIPTION OF THE INVENTION

The present invention provides isolated or purified polypeptides having proteolytic activity and polynucleotides encoding the polypeptides.

Accordingly, the present invention relates to isolated or purified polypeptides having proteolytic activity selected from the group consisting of:

(a) a polypeptide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3, 6, 9, 12, 18 or 21;

(b) a polypeptide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2, 5, 8, 11, 17 or 20;

(c) a polypeptide encoded by a polynucleotide that hybridizes under medium stringency conditions with the full-length complement of the mature polypeptide coding sequence of SEQ ID NO: 1, 4, 7, 10, 16 or 19;

(d) a polypeptide encoded by a polynucleotide having proteolytic and at least 60% sequence identity to the mature polypeptide coding sequence of SEQ ID NO: 1 4, 7, 10, 16 or 19;

(e) a fragment of the polypeptide of (a), (b), (c) or (d) that has proteolytic activity.

The present invention also relates to isolated or purified polynucleotides encoding a signal peptide comprising or consisting of amino acids −26 to −1 of SEQ ID NO: 2, comprising or consisting of amino acids −30 to −1 of SEQ ID NO: 5, comprising or consisting of amino acids −26 to −1 of SEQ ID NO: 8, comprising or consisting of amino acids −41 to −1 of SEQ ID NO: 11, comprising or consisting of amino acids −39 to −1 of SEQ ID NO: 14, comprising or consisting of amino acids −29 to −1 of SEQ ID NO: 17 or comprising or consisting of amino acids −30 to −1 of SEQ ID NO: 20.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the polypeptide shown in SEQ ID NO: 3, which have proteolytic activity. In one aspect, the polypeptides differ by up to 10 amino acids, e.g., 1, 2, 3, 4, 5, 6, 7, 8, 9, or 10, from the polypeptide shown in SEQ ID NO: 3.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the polypeptide shown in SEQ ID NO: 6, which have proteolytic activity. In one aspect, the polypeptides differ by up to 10 amino acids, e.g., 1, 2, 3, 4, 5, 6, 7, 8, 9, or 10, from the polypeptide shown in SEQ ID NO: 6.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the polypeptide shown in SEQ ID NO: 9, which have proteolytic activity. In one aspect, the polypeptides differ by up to 10 amino acids, e.g., 1, 2, 3, 4, 5, 6, 7, 8, 9, or 10, from the polypeptide shown in SEQ ID NO: 9.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the polypeptide shown in SEQ ID NO: 12, which have proteolytic activity. In one aspect, the polypeptides differ by up to 10 amino acids, e.g., 1, 2, 3, 4, 5, 6, 7, 8, 9, or 10, from the polypeptide shown in SEQ ID NO: 12.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the polypeptide shown in SEQ ID NO: 18, which have proteolytic activity. In one aspect, the polypeptides differ by up to 10 amino acids, e.g., 1, 2, 3, 4, 5, 6, 7, 8, 9, or 10, from the polypeptide shown in SEQ ID NO: 18.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the polypeptide shown in SEQ ID NO: 21, which have proteolytic activity. In one aspect, the polypeptides differ by up to 10 amino acids, e.g., 1, 2, 3, 4, 5, 6, 7, 8, 9, or 10, from the polypeptide shown in SEQ ID NO: 21.

In one embodiment the polypeptide preferably comprises, consists essentially of, or consists of the amino acid sequence of SEQ ID NO: 2 or the mature polypeptide thereof; or is a fragment thereof having proteolytic activity. In one aspect, the mature polypeptide is SEQ ID NO: 3.

In one embodiment the polypeptide preferably comprises, consists essentially of, or consists of the amino acid sequence of SEQ ID NO: 5 or the mature polypeptide thereof; or is a fragment thereof having proteolytic activity. In one aspect, the mature polypeptide is SEQ ID NO: 6.

In one embodiment the polypeptide preferably comprises, consists essentially of, or consists of the amino acid sequence of SEQ ID NO: 8 or the mature polypeptide thereof; or is a fragment thereof having proteolytic activity. In one aspect, the mature polypeptide is SEQ ID NO: 9.

In one embodiment the polypeptide preferably comprises, consists essentially of, or consists of the amino acid sequence of SEQ ID NO: 11 or the mature polypeptide thereof; or is a fragment thereof having proteolytic activity. In one aspect, the mature polypeptide is SEQ ID NO: 12.

In one embodiment the polypeptide preferably comprises, consists essentially of, or consists of the amino acid sequence of SEQ ID NO: 17 or the mature polypeptide thereof; or is a fragment thereof having proteolytic activity. In one aspect, the mature polypeptide is SEQ ID NO: 18.

In one embodiment the polypeptide preferably comprises, consists essentially of, or consists of the amino acid sequence of SEQ ID NO: 20 or the mature polypeptide thereof; or is a fragment thereof having proteolytic activity. In one aspect, the mature polypeptide is SEQ ID NO: 21.

In some embodiments, the present invention relates to isolated or purified polypeptides having proteolytic activity encoded by polynucleotides that hybridize under medium stringency conditions, medium-high stringency conditions, high stringency conditions, or very high stringency conditions with the full-length complement of the mature polypeptide coding sequence of SEQ ID NO: 1 or the cDNA thereof (Sambrook et al., 1989, Molecular Cloning, A Laboratory Manual, 2d edition, Cold Spring Harbor, N.Y.).

The polynucleotide of SEQ ID NOs: 1, 4, 7, 10, 13, 16, 19 or a subsequence thereof, as well as the mature polypeptide of SEQ ID NOs: 2, 5, 8, 11, 14, 17, 20 or a fragment thereof, may be used to design nucleic acid probes to identify and clone DNA encoding polypeptides having proteolytic activity from strains of different genera or species according to methods well known in the art. Such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵5, biotin, or avidin). Such probes are encompassed by the present invention.

A genomic DNA or cDNA library prepared from such other strains may be screened for DNA that hybridizes with the probes described above and encodes a polypeptide having proteolytic activity. Genomic or other DNA from such other strains may be separated by agarose or polyacrylamide gel electrophoresis, or other separation techniques. DNA from the libraries or the separated DNA may be transferred to and immobilized on nitrocellulose or another suitable carrier material. In order to identify a clone or DNA that hybridizes with SEQ ID NO: 14, 7, 10, 13, 16, 19 or a subsequence thereof, the carrier material is used in a Southern blot.

For purposes of the present invention, hybridization indicates that the polynucleotides hybridize to a labeled nucleic acid probe corresponding to (i) SEQ ID NOs: 14, 7, 10, 13, 16 or 19; (ii) the mature polypeptide coding sequence of SEQ ID NOs: 14, 7, 10, 13, 16 or 19; (iii) the full-length complement thereof; or (iv) a subsequence thereof; under medium to very high stringency conditions. Molecules to which the nucleic acid probe hybridizes under these conditions can be detected using, for example, X-ray film or any other detection means known in the art.

In some embodiments, the present invention relates to isolated polypeptides having proteolytic activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 1.

In some embodiments, the present invention relates to isolated polypeptides having proteolytic activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 4.

In some embodiments, the present invention relates to isolated polypeptides having proteolytic activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 7.

In some embodiments, the present invention relates to isolated polypeptides having proteolytic activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 10.

In some embodiments, the present invention relates to isolated polypeptides having proteolytic activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 16.

In some embodiments, the present invention relates to isolated polypeptides having proteolytic activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 19.

In one embodiment the polynucleotide encoding the polypeptide preferably comprises, consists essentially of, or consists of nucleotides 79 to 1428 of SEQ ID NO: 1.

In one embodiment the polynucleotide encoding the polypeptide preferably comprises, consists essentially of, or consists of nucleotides 94 to 1137 of SEQ ID NO: 4.

In one embodiment the polynucleotide encoding the polypeptide preferably comprises, consists essentially of, or consists of nucleotides 91 to 1125 of SEQ ID NO: 7.

In one embodiment the polynucleotide encoding the polypeptide preferably comprises, consists essentially of, or consists of nucleotides 124 to 1182 of SEQ ID NO: 10.

In one embodiment the polynucleotide encoding the polypeptide preferably comprises, consists essentially of, or consists of nucleotides 118 to 1323 of SEQ ID NO: 13.

In one embodiment the polynucleotide encoding the polypeptide preferably comprises, consists essentially of, or consists of nucleotides 88 to 1194 of SEQ ID NO: 16.

In one embodiment the polynucleotide encoding the polypeptide preferably comprises, consists essentially of, or consists of nucleotides 91 to 1212 of SEQ ID NO: 19.

In some embodiments, the present invention relates to a polypeptide derived from a mature polypeptide of SEQ ID NO: 3, 6, 9, 12, 15, 18 or 21 by substitution, deletion or addition of one or several amino acids in the mature polypeptide of SEQ ID NO: 3, 6, 9, 12, 15, 18 or 21 respectively. In some embodiments, the present invention relates to variants of the mature polypeptide of SEQ ID NO: 3 comprising a substitution, deletion, and/or insertion at one or more positions. In one aspect, the number of amino acid substitutions, deletions and/or insertions introduced into the mature polypeptide of SEQ ID NO: 3 is up to 10, e.g., 1, 2, 3, 4, 5, 6, 7, 8, 9, or 10. In an embodiment, the polypeptide has an N-terminal extension and/or C-terminal extension of 1-10 amino acids, e.g., 1, 2, 3, 4, 5, 6, 7, 8, 9, or 10 amino acids.

In some embodiments, the present invention relates to variants of the mature polypeptide of SEQ ID NO: 6 comprising a substitution, deletion, and/or insertion at one or more positions. In one aspect, the number of amino acid substitutions, deletions and/or insertions introduced into the mature polypeptide of SEQ ID NO: 6 is up to 10, e.g., 1, 2, 3, 4, 5, 6, 7, 8, 9, or 10. In an embodiment, the polypeptide has an N-terminal extension and/or C-terminal extension of 1-10 amino acids, e.g., 1, 2, 3, 4, 5, 6, 7, 8, 9, or 10 amino acids.

In some embodiments, the present invention relates to variants of the mature polypeptide of SEQ ID NO: 9 comprising a substitution, deletion, and/or insertion at one or more positions. In one aspect, the number of amino acid substitutions, deletions and/or insertions introduced into the mature polypeptide of SEQ ID NO: 9 is up to 10, e.g., 1, 2, 3, 4, 5, 6, 7, 8, 9, or 10. In an embodiment, the polypeptide has an N-terminal extension and/or C-terminal extension of 1-10 amino acids, e.g., 1, 2, 3, 4, 5, 6, 7, 8, 9, or 10 amino acids.

In some embodiments, the present invention relates to variants of the mature polypeptide of SEQ ID NO: 12 comprising a substitution, deletion, and/or insertion at one or more positions. In one aspect, the number of amino acid substitutions, deletions and/or insertions introduced into the mature polypeptide of SEQ ID NO: 12 is up to 10, e.g., 1, 2, 3, 4, 5, 6, 7, 8, 9, or 10. In an embodiment, the polypeptide has an N-terminal extension and/or C-terminal extension of 1-10 amino acids, e.g., 1, 2, 3, 4, 5, 6, 7, 8, 9, or 10 amino acids.

In some embodiments, the present invention relates to variants of the mature polypeptide of SEQ ID NO: 15 comprising a substitution, deletion, and/or insertion at one or more positions.

In one aspect, the number of amino acid substitutions, deletions and/or insertions introduced into the mature polypeptide of SEQ ID NO: 15 is up to 10, e.g., 1, 2, 3, 4, 5, 6, 7, 8, 9, or 10. In an embodiment, the polypeptide has an N-terminal extension and/or C-terminal extension of 1-10 amino acids, e.g., 1, 2, 3, 4, 5, 6, 7, 8, 9, or 10 amino acids.

In some embodiments, the present invention relates to variants of the mature polypeptide of SEQ ID NO: 18 comprising a substitution, deletion, and/or insertion at one or more positions. In one aspect, the number of amino acid substitutions, deletions and/or insertions introduced into the mature polypeptide of SEQ ID NO: 18 is up to 10, e.g., 1, 2, 3, 4, 5, 6, 7, 8, 9, or 10. In an embodiment, the polypeptide has an N-terminal extension and/or C-terminal extension of 1-10 amino acids, e.g., 1, 2, 3, 4, 5, 6, 7, 8, 9, or 10 amino acids.

In some embodiments, the present invention relates to variants of the mature polypeptide of SEQ ID NO: 21 comprising a substitution, deletion, and/or insertion at one or more positions. In one aspect, the number of amino acid substitutions, deletions and/or insertions introduced into the mature polypeptide of SEQ ID NO: 21 is up to 10, e.g., 1, 2, 3, 4, 5, 6, 7, 8, 9, or 10. In an embodiment, the polypeptide has an N-terminal extension and/or C-terminal extension of 1-10 amino acids, e.g., 1, 2, 3, 4, 5, 6, 7, 8, 9, or 10 amino acids.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for proteolytic activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

In some embodiments, the polypeptide is a fragment contains at least 380 amino acid residues (e.g., amino acids 70 to 450 of SEQ ID NO: 3), at least 330 amino acid residues (e.g., amino acids 70 to 400 of SEQ ID NO: 3), at least 205 amino acid residues (e.g., amino acids 240 to 445 of SEQ ID NO: 3), at least 195 amino acid residues (e.g., amino acids 240 to 435 of SEQ ID NO: 3), at least 95 amino acid residues (e.g., amino acids 245 to 340 of SEQ ID NO: 3), or at least 65 amino acid residues (e.g., amino acids 350 to 415 of SEQ ID NO: 3).

In some embodiments, a fragment contains at least 270 amino acid residues (e.g., amino acids 70 to 340 of SEQ ID NO: 6), at least 180 amino acid residues (e.g., amino acids 70 to 250 of SEQ ID NO: 6), at least 265 amino acid residues (e.g., amino acids 75 to 340 of SEQ ID NO: 6), at least 95 amino acid residues (e.g., amino acids 245 to 340 of SEQ ID NO: 6), at least 90 amino acid residues (e.g., amino acids 250 to 340 of SEQ ID NO: 6), or at least 65 amino acid residues (e.g., amino acids 275 to 340 of SEQ ID NO: 6).

In some embodiments, a fragment contains at least 275 amino acid residues (e.g., amino acids 75 to 350 of SEQ ID NO: 9), at least 155 amino acid residues (e.g., amino acids 80 to 235 of SEQ ID NO: 9), at least 152 amino acid residues (e.g., amino acids 81 to 233 of SEQ ID NO:

9), at least 126 amino acid residues (e.g., amino acids 107 to 233 of SEQ ID NO: 9) or at least 70 amino acid residues (e.g., amino acids 270 to 340 of SEQ ID NO: 9).

In some embodiments, a fragment contains at least 180 amino acid residues (e.g., amino acids 70 to 250 of SEQ ID NO: 12), at least 170 amino acid residues (e.g., amino acids 75 to 245 of SEQ ID NO: 12), at least 164 amino acid residues (e.g., amino acids 77 to 241 of SEQ ID NO: 12), at least 285 amino acid residues (e.g., amino acids 70 to 355 of SEQ ID NO: 12) or at least 70 amino acid residues (e.g., amino acids 283 to 351 of SEQ ID NO: 12).

In some embodiments, a fragment contains at least 140 amino acid residues (e.g., amino acids 75 to 215 of SEQ ID NO: 15), at least 265 amino acid residues (e.g., amino acids 80 to 345 of SEQ ID NO: 15), at least 175 amino acid residues (e.g., amino acids 80 to 255 of SEQ ID NO: 15), at least 80 amino acid residues (e.g., amino acids 245 to 325 of SEQ ID NO: 15), at least 85 amino acid residues (e.g., amino acids 260 to 345 of SEQ ID NO: 15) or at least 50 amino acid residues (e.g., amino acids 275 to 325 of SEQ ID NO: 15).

In some embodiments, a fragment contains at least 145 amino acid residues (e.g., amino acids 90 to 235 of SEQ ID NO: 18), at least 160 amino acid residues (e.g., amino acids 90 to 250 of SEQ ID NO: 18), at least 175 amino acid residues (e.g., amino acids 93 to 276 of SEQ ID NO: 18), at least 175 amino acid residues (e.g., amino acids 90 to 265 of SEQ ID NO: 18), at least 85 amino acid residues (e.g., amino acids 270 to 360 of SEQ ID NO: 18), at least 80 amino acid residues (e.g., amino acids 270 to 350 of SEQ ID NO: 18) or at least 55 amino acid residues (e.g., amino acids 295 to 350 of SEQ ID NO: 18).

In some embodiments, a fragment contains at least 180 amino acid residues (e.g., amino acids 90 to 270 of SEQ ID NO: 21), at least 165 amino acid residues (e.g., amino acids 90 to 255 of SEQ ID NO: 21), at least 265 amino acid residues (e.g., amino acids 95 to 360 of SEQ ID NO: 21), at least 85 amino acid residues (e.g., amino acids 265 to 350 of SEQ ID NO: 21), at least 85 amino acid residues (e.g., amino acids 270 to 360 of SEQ ID NO: 21), at least 80 amino acid residues (e.g., amino acids 270 to 350 of SEQ ID NO: 21) or at least 60 amino acid residues (e.g., amino acids 270 to 330 of SEQ ID NO: 21).

Sources of Polypeptides having Proteolytic Activity

A polypeptide having proteolytic activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

In one aspect, the polypeptide is a polypeptide obtained from a Pseudomonas, e.g., a polypeptide obtained from Pseudomonas punonensis.

In another aspect, the polypeptide is a polypeptide obtained from a Psychrobacter, e.g., a polypeptide obtained from Psychrobacter arcticus.

In another aspect, the polypeptide is a polypeptide obtained from a Halomonas, a Cohnella, a Bacillus or a Paenibacillus.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptides may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Nall. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the polynucleotide of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiol. Rev. 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the polypeptide and its source.

In some embodiments, the polypeptide is heterologous to the recombinant host cell.

In some embodiments, at least one of the one or more control sequences is heterologous to the polynucleotide encoding the polypeptide.

In some embodiments, the recombinant host cell comprises at least two copies, e.g., three, four, or five, of the polynucleotide of the present invention.

The host cell may be any microbial or plant cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

Methods of Production The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Halomonas cell. In one aspect, the cell is a Pseudomonas cell. In another aspect, the cell is a Pseudomonas punonensis cell. In one aspect, the cell is a Cohnella cell. In one aspect, the cell is a Bacillus cell. In one aspect, the cell is a Psychrobacter cell. In another aspect, the cell is a Psychrobacter arcticus cell. In one aspect, the cell is a Paenibacillus cell.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid-state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates. The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide

The polypeptide may be recovered using methods known in the art. For example, the polypeptide may be recovered from the fermentation medium by conventional procedures including, but not limited to, collection, centrifugation, filtration, extraction, spray-drying, evaporation, or precipitation. In one aspect, a whole fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

### Cleaning Compositions

In one embodiment, the invention is directed to compositions such as detergent compositions comprising an enzyme of the present invention in combination with one or more additional cleaning composition components. The choice of additional components is within the skill of the artisan and includes conventional ingredients, including the exemplary non-limiting components set forth below under specific headings.

A composition of the invention is preferably a cleaning composition, more preferably a laundry or dishwashing composition. One aspect of the invention relates to a composition comprising a polypeptide of the invention and an adjunct ingredient, wherein the adjunct ingredient is selected from,

a) at least one builder,

b) at least one surfactant, and

c) at least one bleach component.

Some aspects of the invention relate to cleaning compositions e.g. detergent compositions comprising;

a) one or more polypeptide selected from the group consisting of SEQ ID NO: 3, SEQ ID NO: 6, SEQ ID NO: 9, SEQ ID NO: 12, SEQ ID NO: 15, SEQ ID NO: 18 and SEQ ID NO: 21 or a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity hereto, wherein the polypeptide has proteolytic activity, and

b) at least one surfactant, preferably at least one surfactant selected from the group consisting of anionic, non-ionic and/or cationic surfactants.

One aspect of the invention relates to a cleaning composition comprising:
a) at least 0.0001 ppm of active enzyme polypeptide, wherein the enzyme polypeptide is selected from the group consisting of: SEQ ID NO: 3, SEQ ID NO: 6, SEQ ID NO: 9, SEQ ID NO: 12, SEQ ID NO: 15, SEQ ID NO: 18 and SEQ ID NO: 21 or a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto, wherein the polypeptide has proteolytic activity, and
b) from about 2 wt % to about 60 wt % surfactant or from about 5 wt % to about 60 wt % surfactant.

The composition preferably comprises from about 2 wt % to about 60 wt %, from about 5 wt % to about 50 wt %, from about 5 wt % to about 40 wt %, from about 5 wt % to about 30 wt %, from about 5 wt % to about 20 wt %, from about 5 wt % to about 10 wt % anionic surfactants and/or non-ionic surfactants. Preferably, the ratio of anionic/non-ionic surfactant is above 1, i.e. the content of anionic surfactant is higher than the amount of non-ionic surfactant.

One aspect of the invention relates to a detergent composition comprising:
a) at least 0.0001 ppm of active enzyme polypeptide, wherein the polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 3, SEQ ID NO: 6, SEQ ID NO: 9, SEQ ID NO: 12, SEQ ID NO: 15, SEQ ID NO: 18 and SEQ ID NO: 21, wherein the polypeptide has proteolytic activity,
b) from about 5 wt % to about 50 wt % anionic surfactants, and
c) from about 1 wt % to about 20 wt % nonionic surfactants.

The polypeptides of the invention may also be formulated in compositions such as a liquid laundry composition, optionally comprising a builder.

One embodiment relates to a liquid laundry composition comprising:

a) at least 0.0001 ppm of active enzyme polypeptide, selected from the group consisting of SEQ ID NO: 3, SEQ ID NO: 6, SEQ ID NO: 9, SEQ ID NO: 12, SEQ ID NO: 15, SEQ ID NO: 18 and SEQ ID NO: 21 or polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto, wherein the polypeptide has proteolytic activity,

b) from about 2 wt % to about 60 wt % of at least one surfactant, and optionally

c) from about 5 wt % to about 50 wt % of at least one builder such as carbonate, zeolite, phosphate builder, calcium sequestering builder or complexing agents.

The composition comprises at least one builder, wherein the builder is added in an amount from about 0 to about 65% wt %, from about 40 wt % to about 65 wt %, from about 20 wt % to about 65 wt %, from about 10 wt % to about 50 wt % or from about 5 wt % to about 50 wt % weight, wherein the builder is selected among phosphates, sodium citrate builders, sodium carbonate, sodium silicate, sodium and zeolites. Suitable builders are alkali metal or ammonium phosphates, polyphosphates, phosphonates, polyphosphonates, carbonates, bicarbonates, borates, citrates, and polycarboxylates. Citrate builders, e.g., citric acid and soluble salts thereof (particularly sodium salt), are polycarboxylate builders. Citrates can be used in combination with zeolite, silicates like the BRITESIL types, and/or layered silicate builders. The builder is preferably added in an amount of about 0-65% by weight, such as about 5% to about 50% by weight. In the composition, the level of builder is typically about 40-65% by weight, particularly about 50-65% by weight, particularly from 20% to 50% by weight.

One aspect of the invention relates to a cleaning composition comprising:

a) at least 0.0001 ppm active enzyme polypeptide, wherein polypeptide is selected from the group consisting of SEQ ID NO: 3, SEQ ID NO: 6, SEQ ID NO: 9, SEQ ID NO: 12, SEQ ID NO: 15, SEQ ID NO: 18 and SEQ ID NO: 21 or polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto and wherein the polypeptide has proteolytic activity, and optionally

b) from about 2 wt % to about 60 wt % of at least one surfactant, and optionally

c) from about 1 wt % to about 50 wt % of at least one builder, and optionally

d) from about 0.5 wt % to about 20 wt % of at least on bleach component.

The surfactant may be any of those described below under the heading “surfactants”.

In one aspect, the composition is detergent composition such as a laundry composition or an automatic dishwashing (ADW) composition comprising:

a) at least 0.0001 ppm of active enzyme polypeptide, wherein polypeptide is selected from the group consisting of SEQ ID NO: 3, SEQ ID NO: 6, SEQ ID NO: 9, SEQ ID NO: 12, SEQ ID NO: 15, SEQ ID NO: 18 and SEQ ID NO: 21 or polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto, wherein the polypeptide has proteolytic activity, and

b) 2-50 wt % builder selected from citric acid, methylglycine-N, N-diacetic acid (MGDA) and/or glutamic acid-N, N-diacetic acid (GLDA) and mixtures thereof, and optionally

c) at least one bleach component.

The composition may contain 0-30% by weight, such as about 1% to about 20%, such as about 1% to about 10%, such as about 1% to about 5%, such as about 10% to about 30%, such as about 5% to about 10% or such as about 10% to about 20% by weight (wt %) of a bleaching system. Any bleaching system comprising components known in the art for use in cleaning detergents may be utilized. Suitable bleaching system components include sources of hydrogen peroxide; sources of peracids; and bleach catalysts or boosters. Suitable bleach systems are described below under the heading “Bleaching systems”

One aspect of the invention relates to cleaning compositions comprising:
a) at least 0.0001 ppm polypeptide selected from the group consisting of: SEQ ID NO: 3, SEQ ID NO: 6, SEQ ID NO: 9, SEQ ID NO: 12, SEQ ID NO: 15, SEQ ID NO: 18 and SEQ ID NO: 21 or polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto, wherein the polypeptide has proteolytic activity, and optionally
b) from about 2 wt % to about 50 wt % builder preferably selected from citric acid, methylglycine-N, N-diacetic acid (MGDA) and/or glutamic acid-N, N-diacetic acid (GLDA) and mixtures thereof, and optionally
c) from about 2 wt % to about 50 wt % surfactant, preferably selected from anionic surfactants such as LAS, AOS, AEOS and/or non-ionic surfactants such as AE or AEO, and optionally
d) at least one bleach component, preferably selected from percarbonates, persulphates and peracids.
   One aspect of the invention relates to a cleaning composition comprising:
a) a polypeptide having proteolytic activity, wherein polypeptide is selected from the group consisting of SEQ ID NO: 3, SEQ ID NO: 6, SEQ ID NO: 9, SEQ ID NO: 12, SEQ ID NO: 15, SEQ ID NO: 18 and SEQ ID NO: 21 or polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto, and optionally
b) from about 2 wt % to about 50 wt % builder preferably selected from citric acid, methylglycine-N, N-diacetic acid (MGDA) and/or glutamic acid-N, N-diacetic acid (GLDA) and mixtures thereof, and optionally
c) from about 2 wt % to about 60 wt % surfactant, preferably selected from anionic surfactants such as LAS, AOS, AEOS and/or non-ionic surfactants such as AE or AEO, and optionally
d) at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).

One aspect relates to the use of a composition of as described above for cleaning of an item, such as a textile.

A polypeptide of the invention may be included in the cleaning composition of the present invention at a level of from 0.01 to 1000 ppm, from 1 ppm to 1000 ppm, from 10 ppm to 1000 ppm, from 50 ppm to 1000 ppm, from 100 ppm to 1000 ppm, from 150 ppm to 1000 ppm, from 200 ppm to 1000 ppm, from 250 ppm to 1000 ppm, from 250 ppm to 750 ppm, from 250 ppm to 500 ppm.

The concentration of a polypeptide of the invention in the wash liquor solution is typically in the range of wash liquor from 0.00001 ppm to 10 ppm, from 0.00002 ppm to 10 ppm, from 0.0001 ppm to 10 ppm, from 0.0002 ppm to 10 ppm, from 0.001 ppm to 10 ppm, from 0.002 ppm to 10 ppm, from 0.01 ppm to 10 ppm, from 0.02 ppm to 10 ppm, 0.1 ppm to 10 ppm, from 0.2 ppm to 10 ppm, from 0.5 ppm to 5 ppm.

### Surfactants

The cleaning composition may comprise one or more surfactants, which may be anionic and/or cationic and/or non-ionic and/or semi-polar and/or zwitterionic, or a mixture thereof. In a particular embodiment, the detergent composition includes a surfactant system (comprising more than one surfactant) e.g. a mixture of one or more nonionic surfactants and one or more anionic surfactants. In one embodiment the detergent comprises at least one anionic surfactant than at least one non-ionic surfactant, the weight ratio of anionic to nonionic surfactant may be from 10:1 to 1:10. In one embodiment the amount of anionic surfactant is higher than the amount of non-ionic surfactant e.g. the weight ratio of anionic to non-ionic surfactant may be from 10:1 to 1.1:1 or from 5:1 to 1.5:1. The amount of anionic to non-ionic surfactant may also be equal and the weight ratios 1:1. In one embodiment the amount of non-ionic surfactant is higher than the amount of anionic surfactant and the weight ratio may be 1:10 to 1:1.1. Preferably the weight ratio of anionic to non-ionic surfactant is from 10:1 to 1:10, such as from 5:1 to 1:5, or from 5:1 to 1:1.2. Preferably, the weight fraction of non-ionic surfactant to anionic surfactant is from 0 to 0.5 or 0 to 0.2 thus non-ionic surfactant can be present or absent if the weight fraction is 0, but if non-ionic surfactant is present, then the weight fraction of the nonionic surfactant is preferably at most 50% or at most 20% of the total weight of anionic surfactant and non-ionic surfactant. Light duty detergent usually comprises more nonionic than anionic surfactant and there the fraction of non-ionic surfactant to anionic surfactant is preferably from 0.5 to 0.9. The total weight of surfactant(s) is typically present at a level of from about 0.1% to about 60% by weight, such as about 1% to about 40%, or about 3% to about 20%, or about 3% to about 10%.

The one or more surfactants are chosen based on the desired cleaning application and may include any conventional surfactant(s) known in the art. When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, typically available as sodium or potassium salts or salts of monoethanolamine (MEA, 2-aminoethan-1-ol) or triethanolamine (TEA, 2,2′,2″-nitrilotriethan-1-ol); in particular, linear alkylbenzenesulfonates (LAS), isomers of LAS such as branched alkylbenzenesulfonates (BABS) and phenylalkanesulfonates; olefin sulfonates, in particular alpha-olefinsulfonates (AOS); alkyl sulfates (AS), in particular fatty alcohol sulfates (FAS), i.e., primary alcohol sulfates (PAS) such as dodecyl sulfate; alcohol ethersulfates (AES or AEOS or FES, also known as alcohol ethoxysulfates or fatty alcohol ether sulfates); paraffin sulfonates (PS) including alkane-1-sulfonates and secondary alkanesulfonates (SAS); ester sulfonates, including sulfonated fatty acid glycerol esters and alpha-sulfo fatty acid methyl esters (alpha-SFMe or SES or MES); alkyl- or alkenylsuccinic acids such as dodecenyl/tetradecenyl succinic acid (DTSA); diesters and monoesters of sulfosuccinic acid; fatty acid derivatives of amino acids. Furthermore, salts of fatty acids (soaps) may be included.

When included therein the detergent will usually contain from about 1% to about 40% by weight of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADM EAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO) e.g. the AEO-series such as AEO-7, AEO-8 or AEO-9, alcohol propoxylates, in particular propoxylated fatty alcohols (PFA), ethoxylated and propoxylated alcohols, alkoxylated fatty acid alkyl esters, such as ethoxylated and/or propoxylated fatty acid alkyl esters (in particular methyl ester ethoxylates, MEE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0.01 to about 10% by weight of a semipolar surfactant. Non-limiting examples of semipolar surfactants include amine oxides (AO) such as alkyldimethylamine oxides, in particular N-(coco alkyl)-N,N-dimethylamine oxide and N-(tallow-alkyl)-N,N-bis(2-hydroxyethyl)amine oxide, and combinations thereof.

When included therein the detergent will usually contain from about 0.01% to about 10% by weight of a zwitterionic surfactant. Non-limiting examples of zwitterionic surfactants include betaines such as alkyldimethylbetaines, sulfobetaines, and combinations thereof.

Additional bio-based surfactants may be used e.g. wherein the surfactant is a sugar-based non-ionic surfactant which may be a hexyl-β-D-maltopyranoside, thiomaltopyranoside or a cyclic-maltopyranoside, such as described in EP2516606 B1.

### Builders and Co-Builders

The detergent composition may contain about 0-65% by weight, such as about 5% to about 50% of a detergent builder or co-builder, or a mixture thereof. In a dishwashing detergent, the level of builder is typically in the range 40-65%, particularly in the range 50-65%. The builder and/or co-builder may particularly be a chelating agent that forms water-soluble complexes with Ca and Mg. Any builder and/or co-builder known in the art for use in cleaning detergents may be utilized.

Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Clariant), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain from about 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1 hydroxyethane-1,1-diylbis (phosphonic acid (H EDP), Ethylenediaminetetramethylenetetrakis (phosphonic acid) (EDTMPA),diethylenetriaminepentamethylenepentakis (phosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)aspartic acid (SMAS), N-(2-sulfoethyl)aspartic acid (SEAS), N-(2-sulfomethyl)glutamic acid (SMGL), N-(2-sulfoethyl)glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), aminotrimethylenetris(phosphonic acid) (ATMP), and combinations and salts thereof. Further, exemplary builders and/or co-builders are described in, e.g., WO 09/102854 and U.S. Pat. No. 5,977,053.

### Bleaching Systems

The cleaning composition may contain 0-50% by weight, such as 1-40%, such as 1-30%, such as about 1% to about 20%, of a bleaching system. Any oxygen-based bleaching system comprising components known in the art for use in cleaning detergents may be utilized. Suitable bleaching system components include sources of hydrogen peroxide; peracids and sources of peracids (bleach activators); and bleach catalysts or boosters.

Sources of hydrogen peroxide: Suitable sources of hydrogen peroxide are inorganic persalts, including alkali metal salts such as sodium percarbonate and sodium perborates (usually mono- or tetrahydrate), and hydrogen peroxide—urea (1/1). Sources of peracids: Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.

Ad. a) Suitable preformed peracids include, but are not limited to, peroxycarboxylic acids such as peroxybenzoic acid and its ring-substituted derivatives, peroxy-a-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.

Ad. b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

### Bleach Catalysts and Boosters

The bleaching system may also include a bleach catalyst or booster.

Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-κN-methanylylidene)triphenolato-κ3O]manganese(III). The bleach catalysts may also be other metal compounds; such as iron or cobalt complexes.

In some embodiments, where a source of a peracid is included, an organic bleach catalyst or bleach booster may be used having one of the following formulae:

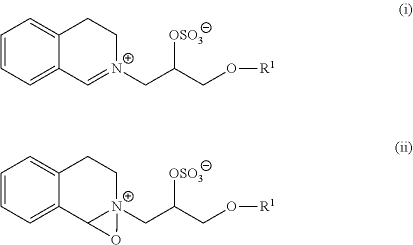

(iii) and mixtures thereof; wherein R1 is independently a branched alkyl group containing from 9 to 24 carbons or linear alkyl group containing from 11 to 24 carbons, preferably R1 is independently a branched alkyl group containing from 9 to 18 carbons or linear alkyl group containing from 11 to 18 carbons, more preferably R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.

Other exemplary bleaching systems are described, e.g. in WO2007/087258, WO2007/087244, WO2007/087259, EP1867708 (Vitamin K) and WO2007/087242.

Suitable photobleaches may for example be sulfonated zinc or aluminium phthalocyanines.

### Polymers

The detergent may contain 0.005-10% by weight, such as 0.5-5%, 2-5%, 0.5-2% or 0.2-1% of a polymer. Any polymer known in the art for use in detergents may be utilized. The polymer may function as a co-builder as mentioned above, or may provide anti-redeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(ethyleneglycol) or poly(ethylene oxide) (PEG or PEO), ethoxylated poly(ethyleneimine), (carboxymethyl)inulin (CMI), carboxylate polymers and polycarboxylates such as polyacrylates, maleic/acrylic acid copolymers, acrylate/styrene copolymers, poly(aspartic) acid, and lauryl methacrylate/acrylic acid copolymers, hydrophobically modified CMC (HM-CMC), silicones, copolymers of terephthalic acid and oligomeric glycols, copolymers of poly(ethylene terephthalate) and poly(oxyethene terephthalate) (PET-POET), poly(vinylpyrrolidone) (PVP), poly(vinylimidazole) (PVI), poly(vinylpyridine-N-oxide) (PVPO or PVPNO) and copoly(vinylimidazole/vinylpyrrolidone) (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Particularly preferred polymer is ethoxylated homopolymer Sokalan® HP 20 from BASF, which helps to prevent redeposition of soil in the wash liquor. Further exemplary polymers include sulfonated polycarboxylates, ethylene oxide-propylene oxide copolymers (PEO-PPO), copolymers of PEG with and vinyl acetate, and diquaternium ethoxy sulfate or quaternized sulfated ethoxylated hexamethylenediamine. Other exemplary polymers are disclosed in, e.g., WO 2006/130575. Salts of the above-mentioned polymers are also contemplated.

### Microorganisms

The detergent additive as well as the detergent composition may also comprise one or more microorganisms, such as one or more fungi, yeast, or bacteria.

In an embodiment, the one or more microorganisms are dehydrated (for example by lyophilization) bacteria or yeast, such as a strain of Lactobacillus.

In another embodiment, the microorganisms are one or more microbial spores (as opposed to vegetative cells), such as bacterial spores; or fungal spores, conidia, hypha. Preferably, the one or more spores are Bacillus endospores; even more preferably the one or more spores are endospores of Bacillus subtilis, Bacillus licheniformis, Bacillus amyloliquefaciens, or Bacillus megaterium.

The microorganisms may be included in the cleaning composition or additive in the same way as enzymes (see above).

### Adjunct Materials

Any cleaning components known in the art for use in cleaning compositions may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in cleaning compositions may be utilized. The choice of such ingredients is well within the skill of the artisan.

### Dispersants

The cleaning compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The cleaning compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The cleaning compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

### Soil Release Polymers

The cleaning compositions of the present invention may also include one or more soil release polymers which aid the removal of soils from fabrics such as cotton and polyester based fabrics, in particular the removal of hydrophobic soils from polyester based fabrics. The soil release polymers may for example be non-ionic or anionic terephthalate-based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymer is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose derivatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, non-ionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The cleaning compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The cleaning compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Additional Enzymes

An enzymatic e.g. cleaning composition of the invention may comprise one or more additional enzymes such as an additional protease, lipase, cutinase, an amylase, carbohydrase, cellulase, pectinase, mannanase, arabinase, galactanase, xylanase, nuclease, oxidase, e.g., a laccase, and/or peroxidase.

In general, the properties of the selected enzyme(s) should be compatible with the selected composition e.g. detergent, (i.e., pH-optimum, compatibility with other enzymatic and non-enzymatic ingredients, etc.), and the enzyme(s) should be present in effective amounts.

### Cellulases

Suitable cellulases include mono-component and mixtures of enzymes of bacterial or fungal origin. Chemically modified or protein engineered mutants are also contemplated. The cellulase may for example be a mono-component or a mixture of mono-component endo-1,4-beta-glucanase also referred to as endoglucanase.

Suitable cellulases include those from the genera Bacillus, Pseudomonas, Humicola, Myceliophthora, Fusarium, Thielavia, Trichoderma, and Acremonium. Exemplary cellulases include a fungal cellulase from Humicola insolens (U.S. Pat. No. 4,435,307) or from Trichoderma, e.g. T. reesei or T. viride. Other suitable cellulases are from Thielavia e.g. Thielavia terrestris as described in WO 96/29397 or the fungal cellulases produced from Myceliophthora thermophila and Fusarium oxysporum disclosed in U.S. Pat. Nos. 5,648,263, 5,691,178, 5,776,757, WO 89/09259 and WO 91/17244. Also relevant are cellulases from Bacillus as described in WO 02/099091 and JP 2000210081. Suitable cellulases are alkaline or neutral cellulases having care benefits. Examples of cellulases are described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Carezyme®, Carezyme® Premium, Celluzyme®, Celluclean®, Celluclast®, Endolase®, Renozyme®; Whitezyme® Celluclean® Classic, Cellusoft® (Novozymes A/S), Puradax®, Puradax HA, and Puradax EG (available from Genencor International Inc.) and KAC-500(B)™ (Kao Corporation).

### Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

### Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases

Suitable amylases which can be used together with the proteolytic polypeptides of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178,

R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+I203Y+G476K

E187P+I203Y+R458N+T459S+D460T+G476K

wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions:

N21D+D97N+V128I

wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases include Duramyl™, Termamyl™, Fungamyl™, Stainzyme™, Stainzyme Plus™, Natalase™, Liquozyme X and BAN™ (from Novozymes A/S), and Rapidase™, Purastar™/Effectenz™, Powerase, Preferenz S1000, Preferenz S100 and Preferenz S110 (from Genencor International Inc./DuPont).

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S). A suitable peroxidase is preferably a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity. Suitable peroxidases also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. The haloperoxidase may be a chloroperoxidase. Preferably, the haloperoxidase is a vanadium haloperoxidase, i.e., a vanadate-containing haloperoxidase. In a preferred method the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis. Haloperoxidases have also been isolated from bacteria such as Pseudomonas, e.g., P. pyrrocinia and Streptomyces, e.g., S. aureofaciens. The haloperoxidase may be derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

Suitable oxidases include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), ora bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885). Suitable examples from bacteria include a laccase derivable from a strain of Bacillus. A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

### Nucleases

Suitable nucleases include deoxyribonucleases (DNases) and ribonucleases (RNases) which are any enzyme that catalyzes the hydrolytic cleavage of phosphodiester linkages in the DNA or RNA backbone respectively, thus degrading DNA and RNA. There are two primary classifications based on the locus of activity. Exonucleases digest nucleic acids from the ends. Endonucleases act on regions in the middle of target molecules. The nuclease is preferably a DNase, which is preferable is obtainable from a microorganism, preferably a fungi or bacterium. In particular, a DNase which is obtainable from a species of Bacillus is preferred; in particular a DNase which is obtainable from Bacillus cibi, Bacillus subtilis or Bacillus licheniformis is preferred. Examples of such DNases are described in WO 2011/098579, WO2014/087011 and WO2017/060475. Particularly preferred is also a DNase obtainable from a species of Aspergillus; in particular a DNase which is obtainable from Aspergillus oryzae, such as a DNase described in WO 2015/155350.

### Proteases

Suitable additional proteases may be of any origin, but are preferably of bacterial or fungal origin, optionally in the form of protein engineered or chemically modified mutants. The protease may be an alkaline protease, such as a serine protease or a metalloprotease. A serine protease may for example be of the S1 family, such as trypsin, or the S8 family such as a subtilisin. A metalloprotease may for example be a thermolysin, e.g. from the M4 family, or another metalloprotease such as those from the M5, M7 or M8 families.

The term “subtilases” refers to a sub-group of serine proteases according to Siezen et al., Protein Eng. 4 (1991) 719-737 and Siezen et al., Protein Sci. 6 (1997) 501-523. Serine proteases are a subgroup of proteases characterized by having a serine in the active site, which forms a covalent adduct with the substrate. The subtilases may be divided into six subdivisions, the Subtilisin family, the Thermitase family, the Proteinase K family, the Lantibiotic peptidase family, the Kexin family and the Pyrolysin family.

Although proteases suitable for detergent use may be obtained from a variety of organisms, including fungi such as Aspergillus, detergent proteases have generally been obtained from bacteria and in particular from Bacillus. Examples of Bacillus species from which subtilases have been derived include Bacillus lentus, Bacillus alkalophilus, Bacillus subtilis, Bacillus amyloliquefaciens, Bacillus licheniformis, Bacillus pumilus and Bacillus gibsonii. Particular subtilisins include subtilisin lentus, subtilisin Novo, subtilisin Carlsberg, subtilisin BPN′, subtilisin 309, subtilisin 147 and subtilisin 168 and e.g. protease PD138 (described in WO 93/18140). Other useful proteases are e.g. those described in WO 01/16285 and WO 02/16547.

Examples of trypsin-like proteases include the Fusarium protease described in WO 94/25583 and WO 2005/040372, and the chymotrypsin proteases derived from Cellumonas described in WO 2005/052161 and WO 2005/052146.

Examples of metalloproteases include the neutral metalloproteases described in WO 2007/044993 such as those derived from Bacillus amyloliquefaciens, as well as e.g. the metalloproteases described in WO 2015/158723 and WO 2016/075078.

Examples of useful proteases are the protease variants described in WO 89/06279 WO 92/19729, WO 96/34946, WO 98/20115, WO 98/20116, WO 99/11768, WO 01/44452, WO 03/006602, WO 2004/003186, WO 2004/041979, WO 2007/006305, WO 2011/036263, WO 2014/207227, WO 2016/087617 and WO 2016/174234. Preferred protease variants may, for example, comprise one or more of the mutations selected from the group consisting of: S3T, V4I, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V102I, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V199I, Q200L, Y203W, S206G, L211Q, L211D, N212D, N212S, M216S, A226V, K229L, Q230H, Q239R, N246K, S253D, N255W, N255D, N255E, L256E, L256D T268A and R269H, wherein position numbers correspond to positions of the Bacillus lentus protease shown in SEQ ID NO: 1 of WO 2016/001449. Protease variants having one or more of these mutations are preferably variants of the Bacillus lentus protease (Savinase®, also known as subtilisin 309) shown in SEQ ID NO: 1 of WO 2016/001449 or of the Bacillus amyloliquefaciens protease (BPN') shown in SEQ ID NO: 2 of WO 2016/001449. Such protease variants preferably have at least 80% sequence identity to SEQ ID NO: 1 or to SEQ ID NO: 2 of WO 2016/001449.

Another protease of interest is the alkaline protease from Bacillus lentus DSM 5483, as described for example in WO 91/02792, and variants thereof which are described for example in WO 92/21760, WO 95/23221, EP 1921147, EP 1921148 and WO 2016/096711.

The protease may alternatively be a variant of the TY145 protease having SEQ ID NO: 1 of WO 2004/067737, for example a variant comprising a substitution at one or more positions corresponding to positions 27, 109, 111, 171, 173, 174, 175, 180, 182, 184, 198, 199 and 297 of SEQ ID NO: 1 of WO 2004/067737, wherein said protease variant has a sequence identity of at least 75% but less than 100% to SEQ ID NO: 1 of WO 2004/067737. TY145 variants of interest are described in e.g. WO 2015/014790, WO 2015/014803, WO 2015/014804, WO 2016/097350, WO 2016/097352, WO 2016/097357 and WO 2016/097354.

Examples of preferred proteases include:

(a) variants of SEQ ID NO: 1 of WO 2016/001449 comprising two or more substitutions selected from the group consisting of S9E, N43R, N76D, Q206L, Y209W, S259D and L262E, for example a variant with the substitutions S9E, N43R, N76D, V205I, Q206L, Y209W, S259D, N261W and L262E, or with the substitutions S9E, N43R, N76D, N185E, S188E, Q191N, A194P, Q206L, Y209W, S259D and L262E, wherein position numbers are based on the numbering of SEQ ID NO: 2 of WO 2016/001449;

(b) a variant of the polypeptide of SEQ ID NO: 1 of WO 2016/001449 with the mutation S99SE, wherein position numbers are based on the numbering of SEQ ID NO: 2 of WO 2016/001449;

(c) a variant of the polypeptide of SEQ ID NO: 1 of WO 2016/001449 with the mutation S99AD, wherein position numbers are based on the numbering of SEQ ID NO: 2 of WO 2016/001449;

(d) a variant of the polypeptide of SEQ ID NO: 1 of WO 2016/001449 with the substitutions Y167A+R170S+A194P, wherein position numbers are based on the numbering of SEQ ID NO: 2 of WO 2016/001449;

(e) a variant of the polypeptide of SEQ ID NO: 1 of WO 2016/001449 with the substitutions S9R+A15T+V68A+N218D+Q245R, wherein position numbers are based on the numbering of SEQ ID NO: 2 of WO 2016/001449;

(f) a variant of the polypeptide of SEQ ID NO: 1 of WO 2016/001449 with the substitutions S9R+A15T+G61E+V68A+A194P+V205I+Q245R+N261D, wherein position numbers are based on the numbering of SEQ ID NO: 2 of WO 2016/001449;

(g) a variant of the polypeptide of SEQ ID NO: 1 of WO 2016/001449 with the substitutions S99D+S101R/E+S103A+V104I+G160S; for example a variant of SEQ ID NO: 1 of WO 2016/001449 with the substitutions S3T+V4I+S99D+S101E+S103A+V104I+G160S+V205I, wherein position numbers are based on the numbering of SEQ ID NO: 2 of WO 2016/001449;

(h) a variant of the polypeptide of SEQ ID NO: 2 of WO 2016/001449 with the substitutions S24G+S53G+S78N+S101N+G128A/S+Y217Q, wherein position numbers are based on the numbering of SEQ ID NO: 2 of WO 2016/001449;

(i) the polypeptide disclosed in GENESEQP under accession number BER84782, corresponding to SEQ ID NO: 302 in WO 2017/210295;

(j) a variant of the polypeptide of SEQ ID NO: 1 of WO 2016/001449 with the substitutions S99D+S101E+S103A+V104I+S156D+G160S+L262E, wherein position numbers are based on the numbering of SEQ ID NO: 2 of WO 2016/001449;

(k) a variant of the polypeptide of SEQ ID NO: 1 of WO 2016/001449 with the substitutions S9R+A15T+G61E+V68A+N76D+S99G+N218D+Q245R, wherein position numbers are based on the numbering of SEQ ID NO: 2 of WO 2016/001449;

(l) a variant of the polypeptide of SEQ ID NO: 1 of WO 2016/001449 with the substitutions V68A+S106A, wherein position numbers are based on the numbering of SEQ ID NO: 2 of WO 2016/001449; and

(m) a variant of the polypeptide of SEQ ID NO: 1 of WO 2004/067737 with the substitutions S27K+N109K+S111E+S171E+S173P+G174K+S175P+F180Y+G182A+L184F+Q198E+N199+T297P, wherein position numbers are based on the numbering of SEQ ID NO: 1 of WO 2004/067737; and

(n) a variant of the polypeptide of SEQ ID NO: 1 of WO 2016/001449 with the substitutions S3T+S9R+R19L+N62D+A194P, wherein position numbers are based on the numbering of SEQ ID NO: 2 of WO 2016/001449.

Suitable commercially available protease enzymes include those sold under the trade names Alcalase®, Duralase™, Durazym™, Relase®, Relase® Ultra, Savinase®, Savinase® Ultra, Primase™, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Blaze Evity® 200T, Neutrase®, Everlase®, Esperase®, Progress® Uno, Progress® In, Progress® Key and Progress® Excel (Novozymes A/S), those sold under the tradename Maxatase™, Maxacal™, Maxapem®, Purafect® Ox, Purafect® OxP, Puramax®, FN2™, FN3™, FN4^{ex}™, Excellase®, Excellenz™ P1000, Excellenz™ P1250, Eraser™, Preferenz® P100, Purafect Prime, Preferenz® P110, Preferenz® P300, Effectenz P1000™, Purafect®, Effectenz P1050™, Purafect® Ox, Effectenz™ P2000, Purafast™, Properase®, Opticlean™ and Optimase® (Danisco/DuPont), BLAP (sequence shown in FIG. 29 of U.S. Pat. No. 5,352,604) and variants hereof (Henkel AG), and KAP (Bacillus alkalophilus subtilisin) from Kao.

### Protease Stabilizers/Inhibitors

The protease of the invention or the proteases as described above, may be stabilized using compounds that act by temporarily reducing the proteolytic activity (reversible inhibitors).

Thus, the composition of the invention may also include a protease inhibitor/stabilizer, which is a reversible inhibitor of protease activity, e.g., serine protease activity. Preferably, the protease inhibitor is a (reversible) subtilisin protease inhibitor. In particular, the protease inhibitor may be a peptide aldehyde, boric acid, or a boronic acid; or a derivative of any of these.

### Boronic Acids

The protease inhibitor may be a boronic acid or a derivative thereof; preferably, a phenylboronic acid or a derivative thereof. In an embodiment of the invention, the phenyl boronic acid derivative is of the following formula:

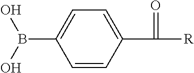

wherein R is selected from the group consisting of hydrogen, hydroxy, C1-C6 alkyl, substituted C1-C6 alkyl, C1-C6 alkenyl and substituted C1-C6 alkenyl. Preferably, R is hydrogen, CH₃, CH₃CH₂ or CH₃CH₂CH₂.

In a preferred embodiment, the protease inhibitor (phenyl boronic acid derivative) is 4 formyl-phenyl boronic acid (4-FPBA).

In another particular embodiment, the protease inhibitor is selected from the group consisting of thiophene-2 boronic acid, thiophene-3 boronic acid, acetamidophenyl boronic acid, benzofuran-2 boronic acid, naphtalene-1 boronic acid, naphtalene-2 boronic acid, 2-FPBA, 3-FBPA, 4-FPBA, 1-thianthrene boronic acid, 4-dibenzofuran boronic acid, 5-methylthiophene-2 boronic, acid, thionaphtrene boronic acid, furan-2 boronic acid, furan-3 boronic acid, 4,4 biphenyl-diborinic acid, 6-hydroxy-2-naphtalene, 4-(methylthio) phenyl boronic acid, 4 (trimethyl-silyl)phenyl boronic acid, 3-bromothiophene boronic acid, 4-methylthiophene boronic acid, 2-naphtyl boronic acid, 5-bromothiphene boronic acid, 5-chlorothiophene boronic acid, dimethylthiophene boronic acid, 2-bromophenyl boronic acid, 3-chlorophenyl boronic acid, 3-methoxy-2-thiophene, p-methyl-phenylethyl boronic acid, 2-thianthrene boronic acid, di-benzothiophene boronic acid, 4-carboxyphenyl boronic acid, 9-anthryl boronic acid, 3,5 dichlorophenyl boronic, acid, diphenyl boronic acidanhydride, o-chlorophenyl boronic acid, p-chlorophenyl boronic acid, m-bromophenyl boronic acid, p-bromophenyl boronic acid, p-flourophenyl boronic acid, p-tolyl boronic acid, o-tolyl boronic acid, octyl boronic acid, 1,3,5 trimethylphenyl boronic acid, 3-chloro-4-flourophenyl boronic acid, 3-aminophenyl boronic acid, 3,5-bis-(triflouromethyl) phenyl boronic acid, 2,4 dichlorophenyl boronic acid, and 4-methoxyphenyl boronic acid.

Further boronic acid derivatives suitable as protease inhibitors in the detergent composition are described in U.S. Pat. Nos. 4,963,655, 5,159,060, WO 95/12655, WO 95/29223, WO 92/19707, WO 94/04653, WO 94/04654, U.S. Pat. Nos. 5,442,100, 5,488,157 and 5,472,628.

### Peptide Aldehyde or Ketone

The protease stabilizer may have the formula: P-A-L-B-BO-R* wherein:

R* is H (hydrogen), CH₃, CX₃, CHX₂, or CH₂X, wherein X is a halogen atom, particularly F (fluorine); preferably, R*═H (so the stabilizer is a peptide aldehyde with the formula P-A-L-B—B0-H);

L is either absent or a linker group of the formula —C(═O)—, —C(═O)—C(═O)—, —C(═S)—, —C(═S)—C(═S)—, or —C(═S)—C(═O)—;

A is absent if L is absent, or is 1 or 2 amino acid residues connected to L via the N-terminal; thus, A may represent A1 or A2-A1, where A2 and A1 each represent one amino acid residue;

B may be 1, 2, or 3 amino acid residues; thus, B may represent B1, B2-B1, or B3-B2-B1, which is connected to B0 via the C-terminal, where B3, B2, and B1 each represent one amino acid residue;

B0 is a single amino acid residue with L- or D-configuration of the formula —NH—CH(R)—C(═O)—;

R is independently selected from the group consisting of C₁₋₆ alkyl, C₆₋₁₀ aryl or C₇₋₁₀ arylalkyl, optionally substituted with one or more, identical or different, substituents R′;

R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂;

R″ is a C₁₋₆ alkyl group; and

P is selected from the group consisting of hydrogen, or—if L is absent—an N-terminal protection group;

B0 may be a single amino acid residue with L- or D-configuration, which is connected to H via the C-terminal of the amino acid. B0 has the formula —NH—CH(R)—C(═O)—, wherein R is a C₁₋₆ alkyl, C₆₋₁₀ aryl or C₇₋₁₀ arylalkyl side chain, such as methyl, ethyl, propyl, isopropyl, butyl, isobutyl, phenyl or benzyl, and wherein R may be optionally substituted with one or more, identical or different, substituents R′. Particular examples of B0 are the D- or L-form of arginine (Arg), 3,4-dihydroxyphenylalanine, isoleucine (Ile), leucine (Leu), methionine (Met), norleucine (Nle), norvaline (Nva), phenylalanine (Phe), m-tyrosine, p-tyrosine (Tyr) and valine (Val). A particular embodiment is when BO is leucine, methionine, phenylalanine, p-tyrosine, or valine. Particularly preferred is p-tyrosine.

B1, which is connected to BO via the C-terminal of the amino acid, may be an aliphatic, hydrophobic and/or neutral amino acid. Examples of B1 are alanine (Ala), cysteine (Cys), glycine (Gly), isoleucine (Ile), leucine (Leu), norleucine (Nle), norvaline (Nva), proline (Pro), serine (Ser), threonine (Thr) and valine (Val). Particular examples of B1 are alanine, glycine, isoleucine, leucine and valine. A particular embodiment is when B1 is alanine, glycine, or valine.

B2, if present, is connected to B1 via the C-terminal of the amino acid, and may be an aliphatic, hydrophobic, neutral and/or polar amino acid. Examples of B2 are alanine (Ala), arginine (Arg), capreomycidine (Cpd), cysteine (Cys), glycine (Gly), isoleucine (Ile), leucine (Leu), norleucine (Nle), norvaline (Nva), phenylalanine (Phe), proline (Pro), serine (Ser), threonine (Thr), and valine (Val). Particular examples of B2 are alanine, arginine, capreomycidine, glycine, isoleucine, leucine, phenylalanine and valine. A particular embodiment is when B2 is arginine, glycine, leucine, phenylalanine, or valine.

B3, if present, is connected to B2 via the C-terminal of the amino acid, and may be a large, aliphatic, aromatic, hydrophobic and/or neutral amino acid. Examples of B3 are isoleucine (Ile), leucine (Leu), norleucine (Nle), norvaline (Nva), phenylalanine (Phe), phenylglycine, tyrosine (Tyr), tryptophan (Trp) and valine (Val). Particular examples of B3 are leucine, phenylalanine, tyrosine, and tryptophan.

The linker group L may be absent or selected from the group consisting of —C(═O)—, —C(═O)—C(═O)—, —C(═S)—, —C(═S)—C(═S)— or —C(═S)—C(═O)—. Particular embodiments of the invention are when L is absent, or L is a carbonyl group —C(═O)—.

A1, if present, is connected to L via the N-terminal of the amino acid, and may be an aliphatic, aromatic, hydrophobic, neutral and/or polar amino acid. Examples of A1 are alanine (Ala), arginine (Arg), capreomycidine (Cpd), glycine (Gly), isoleucine (Ile), leucine (Leu), norleucine (Nle), norvaline (Nva), phenylalanine (Phe), threonine (Thr), tyrosine (Tyr), tryptophan (Trp) and valine (Val). Particular examples of A1 are alanine, arginine, glycine, leucine, phenylalanine, tyrosine, tryptophan and valine. A particular embodiment is when B2 is leucine, phenylalanine, tyrosine or tryptophan.

A2, if present, is connected to Al via the N-terminal of the amino acid, and may be a large, aliphatic, aromatic, hydrophobic and/or neutral amino acid. Examples of A2 are arginine (Arg), isoleucine (Ile), leucine (Leu), norleucine (Nle), norvaline (Nva), phenylalanine (Phe), phenylglycine, Tyrosine (Tyr), tryptophan (Trp) and valine (Val). Particular examples of A2 are phenylalanine and tyrosine.

The N-terminal protection group P (if present) may be selected from formyl, acetyl (Ac), benzoyl (Bz), trifluoroacetyl, methoxysuccinyl, aromatic and aliphatic urethane protecting groups such as fluorenylmethyloxycarbonyl (Fmoc), methoxycarbonyl (Moc), (fluoromethoxy)carbonyl, benzyloxycarbonyl (Cbz), t-butyloxycarbonyl (Boc) and adamantyloxycarbonyl; p-methoxybenzyl carbonyl, benzyl (Bn), p-methoxybenzyl (PMB), p-methoxyphenyl (PMP), methoxyacetyl, methylamino carbonyl, methylsulfonyl, ethylsulfonyl, benzylsulfonyl, methylphosphoramidyl (MeOP(OH)(═O)) and benzylphosphoramidyl (PhCH₂OP(OH)(═O)). Suitable peptide aldehydes are described in WO94/04651, WO95/25791, WO98/13458, WO98/13459, WO98/13460, WO98/13461, WO98/13462, WO07/141736, WO07/145963, WO09/118375, WO10/055052 and WO11/036153. More particularly, the peptide aldehyde may be Cbz-Arg-Ala-Tyr-H, Ac-Gly-Ala-Tyr-H, Cbz-Gly-Ala-Tyr-H, Cbz-Gly-Ala-Tyr-CF₃, Cbz-Gly-Ala-Leu-H, Cbz-Val-Ala-Leu-H, Cbz-Val-Ala-Leu-CF₃, Moc-Val-Ala-Leu-CF₃, Cbz-Gly-Ala-Phe-H, Cbz-Gly-Ala-Phe-CF₃, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, Ac-Leu-Gly-Ala-Tyr-H, Ac-Phe-Gly-Ala-Tyr-H, Ac-Tyr-Gly-Ala-Tyr-H, Ac-Phe-Gly-Ala-Leu-H, Ac-Phe-Gly-Ala-Phe-H, Ac-Phe-Gly-Val-Tyr-H, Ac-Phe-Gly-Ala-Met-H, Ac-Trp-Leu-Val-Tyr-H, MeO—CO-Val-Ala-Leu-H, MeNCO-Val-Ala-Leu-H, MeO—CO-Phe-Gly-Ala-Leu-H, MeO—CO-Phe-Gly-Ala-Phe-H, MeSO₂-Phe-Gly-Ala-Leu-H, MeSO₂-Val-Ala-Leu-H, PhCH₂O—P(OH)(O)-Val-Ala-Leu-H, EtSO₂-Phe-Gly-Ala-Leu-H, PhCH₂SO₂-Val-Ala-Leu-H, PhCH₂O—P(OH)(O)-Leu-Ala-Leu-H, PhCH₂O—P(OH)(O)-Phe-Ala-Leu-H, or MeO—P(OH)(O)-Leu-Gly-Ala-Leu-H. A preferred stabilizer for use in the liquid composition of the invention is Cbz-Gly-Ala-Tyr-H, or a hydrosulfite adduct thereof, wherein Cbz is benzyloxycarbonyl.

Further examples of such peptide aldehydes include α-MAPI, β-MAPI, Phe-C(═O)-Arg-Val-Tyr-H, Phe-C(═O)-Gly-Gly-Tyr-H, Phe-C(═O)-Gly-Ala-Phe-H, Phe-C(═O)-Gly-Ala-Tyr-H, Phe-C(═O)-Gly-Ala-L-H, Phe-C(═O)-Gly-Ala-Nva-H, Phe-C(═O)-Gly-Ala-Nle-H, Tyr-C(═O)-Arg-Val-Tyr-H, Tyr-C(═O)-Gly-Ala-Tyr-H, Phe-C(═S)-Arg-Val-Phe-H, Phe-C(═S)-Arg-Val-Tyr-H, Phe-C(═S)-Gly-Ala-Tyr-H, Antipain, GE20372A, GE20372B, Chymostatin A, Chymostatin B, and Chymostatin C.

The protease stabilizer may be a hydrosulfite adduct of the peptide aldehyde or ketone described above, e.g., as described in WO 2013/004636. The adduct may have the formula P-A-L-B-N(H)—CHR—CH(OH)—SO₃M, wherein P, A, L, B, and R are defined as above, and M is H or an alkali metal, preferably Na or K.

An aqueous solution of the hydrosulfite adduct may be prepared by reacting the corresponding peptide aldehyde with an aqueous solution of sodium bisulfite (sodium hydrogen sulfite, NaHSO₃); potassium bisulfite (KHSO₃) by known methods, e.g., as described in WO 98/47523; U.S. Pat. Nos. 6,500,802; 5,436,229; J. Am. Chem. Soc. (1978) 100, 1228; Org. Synth., Coll. vol. 7: 361.

Particularly preferred peptide aldehyde protease stabilizers have the formula P—B3-B2-B1-B0-H, or a hydrosulfite adduct having the formula P-B3-B2-B1-N(H)—CHR—CHOH—SO₃M, wherein
i) H is hydrogen;
ii) B0 is a single amino acid residue with L- or D-configuration of the formula —NH—CH(R)—C(═O)—;
iii) B1 and B2 are independently single amino acid residues;
iv) B3 is a single amino acid residue, or is absent;
v) R is independently selected from the group consisting of C₁₋₆ alkyl, C₆₋₁₀ aryl or C₇₋₁₀ arylalkyl optionally substituted with one or more, identical or different, substituents R′;
vi) R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂;
vii) R″ is a C₁₋₆ alkyl group;
viii) P is an N-terminal protection group, preferably methoxycarbonyl (Moc) or benzyloxycarbonyl (Cbz); and
ix) M is H or an alkali metal, preferably Na or K.

In an even more preferred embodiment, the peptide aldehyde protease stabilizer has the formula P—B2-B1-B0-H or an adduct having the formula P—B2-B1-N(H)—CHR—CHOH—SO₃M, wherein
i) H is hydrogen;
ii) B0 is a single amino acid residue with L- or D-configuration of the formula —NH—CH(R)—C(═O)—;
iii) B1 and B2 are independently single amino acid residues;
iv) R is independently selected from the group consisting of C₁₋₆ alkyl, C₆₋₁₀ aryl or C₇₋₁₀ arylalkyl optionally substituted with one or more, identical or different, substituents R′;
v) R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂;
vi) R″ is a C₁₋₆ alkyl group;
vii) P is an N-terminal protection group, preferably methoxycarbonyl (Moc) or benzyloxycarbonyl (Cbz); and
viii) M is H or an alkali metal, preferably Na or K.

Preferred embodiments of B0, B1, B2, B3, and P are as described above.

When the peptide aldehyde has the formula P—B3-B2-B1-B0-H, or a hydrosulfite adduct thereof, P is preferably acetyl, methoxycarbonyl, benzyloxycarbonyl, methylamino carbonyl, methylsulfonyl, benzylsulfonyl and benzylphosphoramidyl.

When the peptide aldehyde has the formula P—B2-B1-B0-H, or a hydrosulfite adduct thereof, P is preferably acetyl, methoxycarbonyl, methylsulfonyl, ethylsulfonyl and methylphosphoramidyl.

The molar ratio of the above-mentioned peptide aldehydes (or hydrosulfite adducts) to the protease may be at least 1:1 or 1.5:1, and it may be less than 1000:1, more preferred less than 500:1, even more preferred from 100:1 to 2:1 or from 20:1 to 2:1, or most preferred, the molar ratio is from 10:1 to 2:1.

Formate salts (e.g., sodium formate) and formic acid have also shown good effects as inhibitor of protease activity. Formate can be used synergistically with the above-mentioned protease inhibitors, as shown in WO 2013/004635. The formate salts may be present in the composition in an amount of at least 0.1% w/w or 0.5% w/w, e.g., at least 1.0%, at least 1.2% or at least 1.5%. The amount is typically below 5% w/w, below 4% or below 3%.

In an embodiment, the protease is a metalloprotease and the inhibitor is a metalloprotease inhibitor, e.g., a protein hydrolysate-based inhibitor (e.g., as described in WO 2008/134343).

Formulation of Cleaning e.g. Detergent Products

The cleaning composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multi-compartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water-soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and watersoluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water-soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent , which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Granules

The present invention also relates to enzyme granules/particles comprising the protease i.e. the polypeptide having proteolytic activity of the invention. In an embodiment, the granule comprises a core, and optionally one or more coatings (outer layers) surrounding the core. The core may have a diameter, measured as equivalent spherical diameter (volume based average particle size), of 20-2000 μm, particularly 50-1500 μm, 100-1500 μm or 250-1200 μm. In an embodiment, the core comprises one or more polypeptides having proteolytic activity of the present invention. The core may include additional materials such as fillers, fiber materials (cellulose or synthetic fibers), stabilizing agents, solubilizing agents, suspension agents, viscosity regulating agents, light spheres, plasticizers, salts, lubricants and fragrances. The core may include a binder, such as synthetic polymer, wax, fat, or carbohydrate. The core may include a salt of a multivalent cation, a reducing agent, an antioxidant, a peroxide decomposing catalyst and/or an acidic buffer component, typically as a homogenous blend. The core may include an inert particle with the enzyme absorbed into it, or applied onto the surface, e.g., by fluid bed coating. The core may have a diameter of 20-2000 μm, particularly 50-1500 μm, 100-1500 μm or 250-1200 μm. The core may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA). The coating may be applied in an amount of at least 0.1% by weight of the core, e.g., at least 0.5%, at least 1%, at least 5%, at least 10%, or at least 15%. The amount may be at most 100%, 70%, 50%, 40% or 30%. The coating is preferably at least 0.1 μm thick, particularly at least 0.5 μm, at least 1 μm or at least 5 μm. In some embodiments, the thickness of the coating is below 100 μm, such as below 60 μm, or below 40 μm. The coating should encapsulate the core unit by forming a substantially continuous layer. A substantially continuous layer is to be understood as a coating having few or no holes, so that the core unit it is encapsulating/enclosing has few or none uncoated areas. The layer or coating should, in particular, be homogeneous in thickness. The coating can further contain other materials as known in the art, e.g., fillers, anti-sticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

To provide acceptable protection, the salt coating is preferably at least 0.1 μm thick, e.g., at least 0.5 μm, at least 1 μm, at least 2 μm, at least 4 μm, at least 5 μm, or at least 8 μm. In a particular embodiment, the thickness of the salt coating is below 100 μm, such as below 60 μm, or below 40 μm. The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular, having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminum. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular, alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710.

Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄(CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e. a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄.7H₂O), zinc sulfate heptahydrate (ZnSO₄.7H₂O), sodium phosphate dibasic heptahydrate (Na₂HPO₄.7H₂O), magnesium nitrate hexahydrate (Mg(NO₃)₂(6H₂O)), sodium citrate dihydrate and magnesium acetate tetrahydrate. Preferably the salt is applied as a solution of the salt, e.g., using a fluid bed.

The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB1483591.

The granule may optionally have one or more additional coatings. Examples of suitable coating materials are polyethylene glycol (PEG), methyl hydroxy-propyl cellulose (MHPC) and polyvinyl alcohol (PVA). Examples of enzyme granules with multiple coatings are described in WO 93/07263 and WO 97/23606.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in the Handbook of Powder Technology; Particle size enlargement by C. E. Capes; Volume 1; 1980; Elsevier. Preparation methods include known feed and granule formulation technologies, e.g.,

(a) Spray dried products, wherein a liquid enzyme-containing solution is atomized in a spray drying tower to form small droplets which during their way down the drying tower dry to form an enzyme-containing particulate material. Very small particles can be produced this way (Michael S. Showell (editor); Powdered detergents; Surfactant Science Series; 1998; vol. 71; page 140-142; Marcel Dekker).

(b) Layered products, wherein the enzyme is coated as a layer around a pre-formed inert core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in, e.g., WO 97/23606.

(c) Absorbed core particles, wherein rather than coating the enzyme as a layer around the core, the enzyme is absorbed onto and/or into the surface of the core. Such a process is described in WO 97/39116.

(d) Extrusion or pelletized products, wherein an enzyme-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme (Michael S. Showell (editor); Powdered detergents; Surfactant Science Series; 1998; vol. 71; pages 140-142; Marcel Dekker).

(e) Prilled products, wherein an enzyme-containing powder is suspended in molten wax and the suspension is sprayed, e.g., through a rotating disk atomizer, into a cooling chamber where the droplets quickly solidify (Michael S. Showell (editor); Powdered detergents; Surfactant Science Series; 1998; vol. 71; page 140-142; Marcel Dekker). The product obtained is one wherein the enzyme is uniformly distributed throughout an inert material instead of being concentrated on its surface. U.S. Pat. Nos. 4,016,040 and 4,713,245 describe this technique.

(f) Mixer granulation products, wherein an enzyme-containing liquid is added to a dry powder composition of conventional granulating components. The liquid and the powder in a suitable proportion are mixed and as the moisture of the liquid is absorbed in the dry powder, the components of the dry powder will start to adhere, and agglomerate and particles will build up, forming granulates comprising the enzyme. Such a process is described in U.S. Pat. No. 4,106,991 and related documents EP 170360, EP 304332, EP 304331, WO 90/09440 and WO 90/09428. In a particular product of this process, various high-shear mixers can be used as granulators. Granulates consisting of enzyme, fillers and binders etc. are mixed with cellulose fibers to reinforce the particles to produce a so-called T-granulate. Reinforced particles are more robust, and release less enzymatic dust.

(g) Size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in Martin Rhodes (editor); Principles of Powder Technology; 1990; Chapter 10; John Wiley & Sons.

(h) Fluid bed granulation. Fluid bed granulation involves suspending particulates in an air stream and spraying a liquid onto the fluidized particles via nozzles. Particles hit by spray droplets get wetted and become tacky. The tacky particles collide with other particles and adhere to them to form a granule.

(i) The cores may be subjected to drying, such as in a fluid bed drier. Other known methods for drying granules in the feed or enzyme industry can be used by the skilled person. The drying preferably takes place at a product temperature of from 25 to 90° C. For some enzymes, it is important the cores comprising the enzyme contain a low amount of water before coating with the salt. If water sensitive enzymes are coated with a salt before excessive water is removed, it [what does “it” refer to? Salt?] will be trapped within the core and may affect the activity of the enzyme negatively. After drying, the cores preferably contain 0.1-10% w/w water.

Non-dusting granulates may be produced, e.g., as disclosed in U.S. Pat. Nos. 4,106,991 and 4,661,452 and may optionally be coated by methods known in the art.

The granulate may further one or more additional enzymes. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of enzymes by the use of co-granulates is disclosed in WO 2013/188331.

The present invention also relates to protected enzymes prepared according to the method disclosed in EP 238,216.

In an embodiment, the granule further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, nuclease e.g. DNase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xyloglucanase, xylanase, beta-xylosidase or any combination thereof.

### Liquid Formulations

The present invention also relates to liquid compositions comprising the protease i.e. the polypeptide having proteolytic activity of the invention. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include but are not limited to water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials.

In an aspect, the present invention relates to liquid formulations comprising:

(a) 0.001% to 25% w/w of one or more polypeptides having proteolytic activity of the present invention; and

(b) water.

In another embodiment, the liquid formulation comprises 20% to 80% w/w of polyol. In one embodiment, the liquid formulation comprises 0.001% to 2.0% w/w preservative.

In another embodiment, the invention relates to liquid formulations comprising:

(a) 0.001% to 25% w/w of one or more polypeptides having protease activity of the present invention;

(b) 20% to 80% w/w of polyol;

(c) optionally 0.001% to 2.0% w/w preservative; and

(d) water.

In another embodiment, the invention relates to liquid formulations comprising:

(a) 0.001% to 25% w/w of one or more polypeptides having protease activity of the present invention;

(B) 0.001% to 2.0% w/w preservative;

(C) optionally 20% to 80% w/w of polyol; and

(D) water.

In another embodiment, the liquid formulation comprises one or more formulating agents, such as a formulating agent selected from the group consisting of polyol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the group consisting of sodium sulfate, dextrin, cellulose, sodium thiosulfate, kaolin and calcium carbonate. In one embodiment, the polyols is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600, more preferably selected from the group consisting of glycerol, sorbitol and propylene glycol (MPG) or any combination thereof.

In another embodiment, the liquid formulation comprises 20%-80% polyol (i.e., total amount of polyol), e.g., 25%-75% polyol, 30%-70% polyol, 35%-65% polyol, or 40%-60% polyol. In one embodiment, the liquid formulation comprises 20%-80% polyol, e.g., 25%-75% polyol, 30%-70% polyol, 35%-65% polyol, or 40%-60% polyol, wherein the polyol is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20%-80% polyol (i.e., total amount of polyol), e.g., 25%-75% polyol, 30%-70% polyol, 35%-65% polyol, or 40%-60% polyol, wherein the polyol is selected from the group consisting of glycerol, sorbitol and propylene glycol (MPG).

In another embodiment, the preservative is selected from the group consisting of sodium sorbate, potassium sorbate, sodium benzoate and potassium benzoate or any combination thereof. In one embodiment, the liquid formulation comprises 0.02% to 1.5% w/w preservative, e.g., 0.05% to 1.0% w/w preservative or 0.1% to 0.5% w/w preservative. In one embodiment, the liquid formulation comprises 0.001% to 2.0% w/w preservative (i.e., total amount of preservative), e.g., 0.02% to 1.5% w/w preservative, 0.05% to 1.0% w/w preservative, or 0.1% to 0.5% w/w preservative, wherein the preservative is selected from the group consisting of sodium sorbate, potassium sorbate, sodium benzoate and potassium benzoate or any combination thereof.

In another embodiment, the liquid formulation further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof.

### Fermentation Broth Formulations or Cell Compositions

The present invention also relates to a fermentation broth formulation or a cell composition comprising a polypeptide having proteolytic activity of the present invention. The fermentation broth formulation or the cell composition further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In some embodiments, the fermentation broth formulation or the cell composition comprises a first organic acid component comprising at least one 1-5 carbon organic acid and/or a salt thereof and a second organic acid component comprising at least one 6 or more carbon organic acid and/or a salt thereof. In some embodiments, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In some embodiments, the killed cells and/or cell debris are removed from a cell-killed whole broth to provide a composition that is free of these components.

The fermentation broth formulation or cell composition may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells.

In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell composition of the present invention may be produced by a method described in WO 90/15861 or WO 2010/096673.

### Uses

The polypeptides of the present invention may be added to and thus become a component of a detergent composition. The cleaning composition may be formulated, for example, as a hand or machine laundry detergent composition including a laundry additive composition suitable for pre-treatment of stained fabrics and a rinse added fabric softener composition or be formulated as a detergent composition for use in general household hard surface cleaning operations or be formulated for hand or machine dishwashing operations.

In one aspect, the present invention relates to a cleaning composition comprising a polypeptide of the present invention as described herein.

One aspect of the invention relates to the use of a polypeptide of the invention for preventing, reducing or removing the adherence of soil to an item. In one embodiment, the item is textile. When the soil does not adhere to the item, the item appears cleaner. Thus, the invention further concerns the use of a polypeptide having proteolytic activity of the invention for maintaining or improving the whiteness of the item.

When items like T-shirts or sportswear are used, they are exposed to bacteria from the body of the user and from the rest of the environment in which they are used. This may cause malodour on the item even after the item is washed. The present invention therefore also concerns removal or reduction of malodour on textile. The malodour may be caused by bacteria producing compounds with an unpleasant smell. One example of such unpleasant smelling compounds is E-2-nonenal. The malodour can be present on newly washed textile which is still wet. Or the malodour can be present on newly washed textile, which has subsequently been dried. The malodour may also be present on textile, which has been stored for some time after wash. The present invention relates to reduction or removal of malodour such as E-2-nonenal from wet or dry textile.

One aspect of the invention relates to the use of a polypeptide having proteolytic activity, wherein the polypeptide having at 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptides shown in SEQ ID NO: 3, SEQ ID NO: 6, SEQ ID NO: 9, SEQ ID NO: 12, SEQ ID NO: 15, SEQ ID NO: 18 or SEQ ID NO: 21,

(i) for preventing, reducing or removing stickiness of the item;

(ii) for pretreating stains on the item;

(iii) for preventing, reducing or removing redeposition of soil during a wash cycle;

(iv) for preventing, reducing or removing adherence of soil to the item;

(v) for maintaining or improving whiteness of the item;

(vi) for preventing, reducing or removal malodor from the item,
wherein the item is a textile.

The invention is further defined by the following numbered paragraphs:

1. An isolated or purified polypeptide having proteolytic activity, selected from the group consisting of:

(a) a polypeptide having at least 60% sequence identity to SEQ ID NO: 2, 5, 8, 11, 14, 17 or 20;

(b) a polypeptide having at least 60% sequence identity to the sequence of SEQ ID NO: 3, 6, 9, 12, 15, 18 or 21;

(c) a polypeptide having at least 60% sequence identity to a mature polypeptide of SEQ ID NO: 2, 5, 8, 11, 14, 17 or 20;

(d) a polypeptide encoded by a polynucleotide that hybridizes under high stringency conditions with the full-length complement of the mature polypeptide coding sequence of SEQ ID NO: 1, 4, 7, 10, 13, 16 or 19;

(e) a polypeptide encoded by a polynucleotide having at least 60% sequence identity to the mature polypeptide coding sequence of SEQ ID NO: 1, 4, 7, 10, 13, 16 or 19;

(f) a polypeptide derived from a mature polypeptide of SEQ ID NO: 3, 6, 9, 12, 15, 18 or 21 by substitution, deletion or addition of one or several amino acids in the mature polypeptide of 3, 6, 9, 12, 15, 18 or 21 respectively; and

(g) a fragment of the polypeptide of (a), (b), (c), (d), (e) or (f) that has proteolytic activity.
2. An isolated or purified polypeptide having proteolytic activity, selected from the group consisting of:

(a) a polypeptide having at least 60% sequence identity to SEQ ID NO: 2, 5, 8, 11, 14, 17 or 20;

(b) a polypeptide having at least 60% sequence identity to SEQ ID NO: 3, 6, 9, 12, 15, 18 or 21;

(c) a polypeptide encoded by a polynucleotide that hybridizes under high stringency conditions with the full-length complement of the mature polypeptide coding sequence of SEQ ID NO: 1, 4, 7, 10, 13, 16 or 19;

(d) a polypeptide encoded by a polynucleotide having at least 60% sequence identity to the mature polypeptide coding sequence of SEQ ID NO: 1, 4, 7, 10, 13, 16 or 19; and

(e) a fragment of the polypeptide of (a), (b), (c), or (d), that has proteolytic activity.
3. An isolated or purified polypeptide having proteolytic activity, which is:

(a) a polypeptide having at least 60% sequence identity to SEQ ID NO: 2, 5, 8, 11, 14, 17 or 20; or

(b) a fragment of the polypeptide of (a), that has proteolytic activity.
4. An isolated or purified polypeptide having proteolytic activity, which is:

(a) a polypeptide having at least 60% sequence identity to SEQ ID NO: 3, 6, 9, 12, 15, 18 or 21; or

(b) a fragment of the polypeptide of (a), that has proteolytic activity.
5. An isolated or purified polypeptide having proteolytic activity, which is:

(a) a polypeptide having at least 60% sequence identity to a mature polypeptide of SEQ ID NO: 2, 5, 8, 11, 14, 17 or 20; or

(B) a fragment of the polypeptide of (a), that has proteolytic activity.
6. An isolated or purified polypeptide having proteolytic activity, which is a fragment selected from the group consisting of:
   a) a fragment of SEQ ID NO: 3, wherein the fragment preferably contains at least 380 amino acid residues (e.g., amino acids 70 to 450 of SEQ ID NO: 3), at least 205 amino acid residues (e.g., amino acids 240 to 445 of SEQ ID NO: 3), at least 195 amino acid residues (e.g., amino acids 240 to 445 of SEQ ID NO: 3), at least 95 amino acid residues (e.g., amino acids 245 to 340 of SEQ ID NO: 3), or at least 65 amino acid residues (e.g., amino acids 350 to 415 of SEQ ID NO: 3) and wherein the fragment has proteolytic activity,
   b) a fragment of SEQ ID NO: 6, wherein the fragment preferably contains at least 270 amino acid residues (e.g., amino acids 70 to 340 of SEQ ID NO: 6), at least 180 amino acid residues (e.g., amino acids 70 to 250 of SEQ ID NO: 6), at least 265 amino acid residues (e.g., amino acids 75 to 340 of SEQ ID NO: 6), at least 95 amino acid residues (e.g., amino acids 245 to 340 of SEQ ID NO: 6), at least 90 amino acid residues (e.g., amino acids 250 to 340 of SEQ ID NO: 6), or at least 65 amino acid residues (e.g., amino acids 275 to 340 of SEQ ID NO: 6) and wherein the fragment has proteolytic activity,
   c) a fragment of SEQ ID NO: 9, wherein the fragment preferably contains at least 275 amino acid residues (e.g., amino acids 75 to 350 of SEQ ID NO: 9), at least 155 amino acid residues (e.g., amino acids 80 to 235 of SEQ ID NO: 9), at least 152 amino acid residues (e.g., amino acids 81 to 233 of SEQ ID NO: 9), at least 126 amino acid residues (e.g., amino acids 107 to 233 of SEQ ID NO: 9) or at least 70 amino acid residues (e.g., amino acids 270 to 340 of SEQ ID NO: 9) and wherein the fragment has proteolytic activity,
   d) a fragment of SEQ ID NO: 12, wherein the fragment preferably contains at least 180 amino acid residues (e.g., amino acids 70 to 250 of SEQ ID NO: 12), at least 170 amino acid residues (e.g., amino acids 75 to 245 of SEQ ID NO: 12), at least 164 amino acid residues (e.g., amino acids 77 to 241 of SEQ ID NO: 12), at least 285 amino acid residues (e.g., amino acids 70 to 355 of SEQ ID NO: 12) or at least 70 amino acid residues (e.g., amino acids 283 to 351 of SEQ ID NO: 12) and wherein the fragment has proteolytic activity,
   e) a fragment of SEQ ID NO: 15, wherein the fragment preferably contains at least 140 amino acid residues (e.g., amino acids 75 to 215 of SEQ ID NO: 15), at least 265 amino acid residues (e.g., amino acids 80 to 345 of SEQ ID NO: 15), at least 175 amino acid residues (e.g., amino acids 80 to 255 of SEQ ID NO: 15), at least 80 amino acid residues (e.g., amino acids 245 to 325 of SEQ ID NO: 15), at least 85 amino acid residues (e.g., amino acids 260 to 345 of SEQ ID NO: 15) or at least 50 amino acid residues (e.g., amino acids 275 to 325 of SEQ ID NO: 15) and wherein the fragment has proteolytic activity,
   f) a fragment of SEQ ID NO: 18, wherein the fragment preferably contains at least 145 amino acid residues (e.g., amino acids 90 to 235 of SEQ ID NO: 18), at least 160 amino acid residues (e.g., amino acids 90 to 250 of SEQ ID NO: 18), at least 175 amino acid residues (e.g., amino acids 93 to 276 of SEQ ID NO: 18), at least 175 amino acid residues (e.g., amino acids 90 to 265 of SEQ ID NO: 18), at least 85 amino acid residues (e.g., amino acids 270 to 360 of SEQ ID NO: 18), at least 80 amino acid residues (e.g., amino acids 270 to 350 of SEQ ID NO: 18) or at least 55 amino acid residues (e.g., amino acids 295 to 350 of SEQ ID NO: 18) and wherein the fragment has proteolytic activity, and
   g) a fragment of SEQ ID NO: 21, wherein the fragment preferably contains at least 180 amino acid residues (e.g., amino acids 90 to 270 of SEQ ID NO: 21), at least 165 amino acid residues (e.g., amino acids 90 to 255 of SEQ ID NO: 21), at least 265 amino acid residues (e.g., amino acids 95 to 360 of SEQ ID NO: 21), at least 85 amino acid residues (e.g., amino acids 265 to 350 of SEQ ID NO: 21), at least 85 amino acid residues (e.g., amino acids 270 to 360 of SEQ ID NO: 21), at least 80 amino acid residues (e.g., amino acids 270 to 350 of SEQ ID NO: 21) or at least 60 amino acid residues (e.g., amino acids 270 to 330 of SEQ ID NO: 21).
7. The polypeptide of any one of paragraphs 1-6, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 2, 5, 8, 11, 14, 17 or 20.
8. The polypeptide of any one of paragraphs 1-7, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 3, 6, 9, 12, 15, 18 or 21.
9. The polypeptide of any one of paragraphs 1-8, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to a mature polypeptide of SEQ ID NO: 2, 5, 8, 11, 14, 17 or 20.
10. The polypeptide of any one of paragraphs 1-9, which is encoded by a polynucleotide that hybridizes under medium stringency conditions, medium-high stringency conditions, high stringency conditions, or very high stringency conditions with the full-length complement of the mature polypeptide coding sequence of SEQ ID NO: 1, 4, 7, 10, 13, 16 or 19 or the cDNA thereof.
11. The polypeptide of any one of paragraphs 1-10, which is encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 1, 4, 7, 10, 13, 16 or 19.
12. The polypeptide of any one of paragraphs 1-10, which is a variant of SEQ ID NO: 3, 6, 9, 12, 15, 18 or 21 comprising a substitution, deletion, and/or insertion at one or more positions.
13. The polypeptide of any one of paragraphs 1-10, comprising, consisting essentially of, or consisting of SEQ ID NO: 2, 5, 8, 11, 14, 17 or 20.
14. The polypeptide of any one of paragraphs 1-10, comprising, consisting essentially of, or consisting of SEQ ID NO: 3, 6, 9, 12, 15, 18 or 21.
15. The polypeptide of any one of paragraphs 1-10, comprising SEQ ID NO: 3, 6, 9, 12, 15, 18 or 21 and an N-terminal extension and/or C-terminal extension of 1-10 amino acids, e.g., 1, 2, 3, 4, 5, 6, 7, 8, 9, or 10 amino acids.
16. The polypeptide of any one of paragraphs 1-10, which is a mature polypeptide of SEQ ID NO: 2, 5, 8, 11, 14, 17 or 20.
17. A fusion polypeptide comprising the polypeptide of any one of paragraphs 1-16 and a second polypeptide.
18. A granule, which comprises:

(a) a core comprising the polypeptide of any one of paragraphs 1-16, and optionally,

(b) a coating consisting of one or more layer(s) surrounding the core.
19. A granule, which comprises:

(a) a core, and

(b) a coating consisting of one or more layer(s) surrounding the core, wherein the coating comprises the polypeptide of any one of paragraphs 1-16.
20. A composition comprising the polypeptide of any one of paragraphs 1-16 or the granule of paragraph 18 or 19.
21. The composition of claim 20, wherein the composition is a cleaning composition, comprising at least one cleaning component.
22. A whole broth formulation or cell culture composition comprising the polypeptide of any one of paragraphs 1-16.
23. An isolated or purified polynucleotide encoding the polypeptide of any one of paragraphs 1-16.
24. The polynucleotide of claim 23, which comprises SEQ ID NO: 1 or nucleotides 79 to 1428 of SEQ ID NO: 1, SEQ ID NO: 4 or nucleotides 94 to 1137 of SEQ ID NO: 4, SEQ ID NO: 7 or nucleotides 91 to 1125 of SEQ ID NO: 7, SEQ ID NO: 10 or nucleotides 124 to 1182 of SEQ ID NO: 10, SEQ ID NO: 13 or nucleotides 118 to 1323 of SEQ ID NO: 13, SEQ ID NO: 16 or nucleotides 88 to 1194 of SEQ ID NO: 16 or SEQ ID NO: 19 or nucleotides 91 to 1212 of SEQ ID NO: 19.
25. A nucleic acid construct or expression vector comprising the polynucleotide of paragraph 23 or 24, wherein the polynucleotide is operably linked to one or more control sequences that direct the production of the polypeptide in an expression host.
26. A recombinant host cell comprising the polynucleotide of paragraph 23 or 24 operably linked to one or more control sequences that direct the production of the polypeptide.
27. The recombinant host cell of paragraph 26, wherein the polypeptide is heterologous to the recombinant host cell.
28. The recombinant host cell of paragraph 26 or 27, wherein at least one of the one or more control sequences is heterologous to the polynucleotide encoding the polypeptide.
29. The recombinant host cell of any one of paragraphs 26-28, which comprises at least two copies, e.g., three, four, or five, of the polynucleotide of paragraph 23 or 24.
30. The recombinant host cell of any one of paragraphs 26-29, which is a yeast recombinant host cell, e.g., a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.
31. The recombinant host cell of any one of paragraphs 26-29, which is a filamentous fungal recombinant host cell, e.g., an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell, in particular, an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.
32. The recombinant host cell of any one of paragraphs 26-29, which is a prokaryotic recombinant host cell, e.g., a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, or a Gram-negative bacteria selected from the group consisting of Campylobacter, E. coli, Flavobacterium, Fusobacterium, Helicobacter, Ilyobacter, Neisseria, Pseudomonas, Salmonella, and Ureaplasma cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. Zooepidemicus, Streptomyces achromogenes, Streptomyces avermitilis, Streptomyces coelicolor, Streptomyces griseus, and Streptomyces lividans cells.
33. A method of producing the polypeptide of any one of paragraphs 1-16, comprising cultivating a cell, which in its wild-type form produces the polypeptide, under conditions conducive for production of the polypeptide.
34. The method of paragraph 33, further comprising recovering the polypeptide.
35. A method of producing a polypeptide having proteolytic activity, comprising cultivating the recombinant host cell of any one of paragraphs 26-32 under conditions conducive for production of the polypeptide.
36. The method of paragraph 35, further comprising recovering the polypeptide.
37. Use of a polypeptide of any of paragraphs 1-16, a fusion protein of claim 17 or a granule of claim 18 or 19 for cleaning of an item, wherein the item is a textile or a hard surface.
38. Use according to paragraph 37, wherein the polypeptide is used in a cleaning process, such as laundry and/or dishwashing.
39. Use according to any of paragraphs 37 or 38, wherein the item is a textile.
40. Use according to any of paragraphs 37 to 39 for preventing, reducing or removing stickiness of the item.
41. Use according to any of paragraphs 37 to 40 for preventing, reducing or removing malodour of the item.
42. Use according to any of paragraphs 37 to 40 for preventing, reducing or removing redeposition of soil during a wash cycle.
43. A method of cleaning an item, comprising the steps of:
a) exposing an item to a wash liquor comprising a polypeptide of any one of paragraphs 1-16 or a cleaning composition comprising said polypeptide;
b) completing at least one wash cycle; and
optionally rinsing the item,
wherein the item is a textile or a hard surface.

Note that the singular forms “a,” “an,” and “the” include plural references unless the context clearly dictates otherwise.

Reference to “about” a value or parameter herein includes aspects that are directed to that value or parameter per se. For example, description referring to “about X” includes the aspect “X”.

Unless defined otherwise or clearly indicated by context, all technical and scientific terms used herein have the same meaning as commonly understood by one of ordinary skill in the art to which this invention belongs.

The invention is further described in the following non-limited examples.

### EXAMPLES

Microorganism strains

| Strain | Sequence | Country of origin |
|---|---|---|
| Halomonas sp. | SEQ ID NO: 3 | United States |
| Pseudomonas punonensis | SEQ ID NO: 6 | Denmark |
| Cohnella sp. | SEQ ID NO: 9 | Denmark |
| Bacillus sp. | SEQ ID NO: 12 | Australia |
| Psychrobacter arcticus | SEQ ID NO: 15 | SWISSPROT: Q1QBQ7 |
| Paenibacillus sp. | SEQ ID NO: 18 | United States |
| Halomonas sp. | SEQ ID NO: 21 | United States |

### Assays

### Assay I: Testing of Protease Activity

Proteolytic activity can be determined by a method employing Suc-AAPF-PNA as the substrate. Suc-AAPF-PNA is an abbreviation for N-Succinyl-Alanine-Alanine-Proline-Phenylalanine-p-Nitroanilide and is a blocked peptide which can be cleaved by endo-proteases. Following cleavage, a free PNA molecule is liberated, which has a yellow color and thus can be measured by visible spectrophotometry at wavelength 405 nm. The Suc-AAPF-PNA substrate is manufactured by Bachem (cat. no. L1400, dissolved in DMSO). The protease sample to be analyzed is diluted in residual activity buffer (100 mM Tris pH 8.6). The assay is performed by transferring 3 0 μl of diluted enzyme samples to 96 well microtiter plate and adding 70 μl substrate working solution (0.72 mg/ml in 100 mM Tris pH8.6). The solution was mixed at room temperature and absorption is measured every 20 seconds over 5 minutes at OD 405 nm. The slope (absorbance per minute) of the time dependent absorption-curve is directly proportional to the activity of the protease in question under the given set of conditions. The protease sample is diluted to a level where the slope is linear .

### Automatic Mechanical Stress Assay (AMSA) for Laundry

In order to assess the wash performance in laundry, washing experiments are performed using the Automatic Mechanical Stress Assay (AMSA). With the AMSA, the wash performance of many small volume enzyme-detergent solutions can be examined. The AMSA plate has a number of slots for test solutions and a lid that firmly squeezes the textile to be washed against the slot openings. During the wash, the plate, test solutions, textile and lid are vigorously shaken to bring the test solution in contact with the textile and apply mechanical stress in a regular, periodic, oscillating manner. For further description see WO02/42740 especially the paragraph “Special method embodiments” at page 23-24.

The laundry experiments are typically conducted under the experimental conditions specified below:

| | |
|---|---|
| Detergent dosage | 5 g/L (liquid detergent) |
| | 2.5 g/L (powder detergent) |
| Test solution volume | 160 micro L |
| pH | Adjusted to pH 7 or pH 6 (liquid detergent) |
| | As is (powder detergent) |
| Wash time | 20 minutes |
| Temperature | 60° C., 40° C. and 20° C. or 15° C. |
| Water hardness | 15° dH |

### Examples of Model Detergents

| | |
|---|---|
| Laundry liquid | Sodium alkylethoxy sulfate (C-9-15, 2EO) 6.0% |
| model detergent | Sodium dodecyl benzene sulfonate 3.0% |
| | Sodium toluene sulfonate 3.0% |
| | Oleic acid 2.0% |
| | Primary alcohol ethoxylate (C12-15, 7EO) 3.0% |
| | Primary alcohol ethoxylate (C12-15, 3EO) 2.5% |
| | Ethanol 0.5% |
| | Monopropylene glycol 2.0% |
| | Tri-sodium citrate dihydrate 4.0% |
| | Triethanolamine 0.4% |
| | De-ionized water ad 100% |
| | pH adjusted to 8.5 with NaOH |
| Laundry powder | Sodium citrate dihydrate 32.3% |
| model detergent | Sodium-LAS 24.2% |
| | Sodium lauryl sulfate 32.2% |
| | Neodol 25-7 (alcohol ethoxylate) 6.4% |
| | Sodium sulfate 4.9% |

Test materials are obtained from EMPA Testmaterials AG, Mövenstrasse 12, CH-9015 St. Gallen, Switzerland, from Center for Testmaterials BV, P.O. Box 120, 3133 KT Vlaardingen, the Netherlands, and WFK Testgewebe GmbH, Christenfeld 10, D-41379 Bruggen, Germany.

Water hardness is adjusted to 15° dH by addition of CaCl₂, MgCl₂, and NaHCO₃ (Ca²⁺:Mg²⁺=4:1:7.5) to the test system. After washing the textiles are flushed in tap water and dried.

The wash performance is measured as the brightness of the colour of the textile washed. Brightness can also be expressed as the intensity of the light reflected from the sample when illuminated with white light. When the sample is stained the intensity of the reflected light is lower, than that of a clean sample. Therefore, the intensity of the reflected light can be used to measure wash performance. Colour measurements are made with a professional flatbed scanner (Kodak iQsmart, Kodak, Midtager 29, DK-2605 Brøndby, Denmark), which is used to capture an image of the washed textile.

To extract a value for the light intensity from the scanned images, 24-bit pixel values from the image are converted into values for red, green and blue (RGB). The intensity value (Int) is calculated by adding the RGB values together as vectors and then taking the length of the resulting vector:

Int=\sqrt{r²+g²+b²}

### Swatch Sample Staining and Imaging

Biofilm swatch samples for testing are stored refrigerated in plastic bags before use. The samples are stained by placing them in a tray containing phosphate buffered saline, 0.025% Tween 20, 0.1% Triton X 100, and fluorescent dye diluted to a final concentration of 5 μM. Different fluorescent dyes used may be used for this method depending on the substrate, e.g. SYTO™ BC DNA dye, FUN™ 1 DNA/metabolic dye or Fluo-4 calcium binding dye. DNA staining provides an indication of residual organism on the swatches, whereas protein staining using, for example, Sypro™ Ruby Red protein stain may be used for direct detection of residual protein. The samples are incubated at room temperature for 30-60 minutes for staining. After incubation, the dye solution is removed and the samples are washed 2-3 times with PBS, 0.025% Tween 20 and 0.1% Triton X 100. After washing, samples are transferred to a clear bottom, black walled 96-well imaging plate, and a 3-D printed plastic piece is placed on top to hold each swatch sample flat to the bottom of the sample well. Digital Max projection images at 4× or 10× magnification are collected for each sample using a GE IN Cell 2200 automated imager. Individual images are analyzed using GE Developer image analysis software, and the area detected is used for quantification of specific biomolecule staining, with a lower stained area indicating reduced staining and thus reduced residual substrate. Statistical analysis is performed in JMP, version 10.

### Example 1

### Cloning and Expression

The peptidases were derived from bacterial strains isolated from environmental soil samples by standard microbiological isolation techniques. The isolated pure strains were identified, and taxonomy was assigned based on DNA sequencing of the 16S ribosomal genes.

Chromosomal DNA was isolated from pure culture with the DNeasy Blood & Tissue Kit from Qiagen (Hilden, Germany) and subjected to full genome sequencing using Illumina technology. Genome sequencing, the subsequent assembly of reads and the gene discovery (i.e. annotation of gene functions) is known to the person skilled in the art and the service can be purchased commercially.

The genome sequence was analyzed for putative peptidases from the MEROPS database family S1.174 (Rawlings, N. D. et al.(2018) The MEROPS database of proteolytic enzymes, their substrates and inhibitors in 2017 and a comparison with peptidases in the PANTHER database (Nucleic Acids Res 2018, 46, D624-D632). This analysis identified a gene encoding a putative peptidase which was subsequently cloned and recombinantly expressed in Bacillus subtilis.

The natural gene encoding the peptidase was codon optimized for expression in Bacillus subtilis and in addition the coding region of the native signal peptide was replaced by a sequence encoding the Bacillus clausii secretion signal (encoding the following amino acid sequence: MKKPLGKIVASTALLISVAFSSSIASA (SEQ ID NO: 22) and finally a sequence encoding a C-terminal histidine affinity tag (encoding the following amino acid sequence: HHHHHH (SEQ ID NO: 23) was added to ease downstream purification. The resulting gene was ordered as a fully synthetically produced DNA fragment from Twist Bioscience (San Francisco, Calif., USA)

The synthetic gene was subcloned into an E. coli vector and the resulting plasmid was transformed into a natural competent Bacillus subtilis host where the gene integrates by double cross over homologues recombination into the pectate lyase locus of the B. subtilis genome, described in patent application WO 2003/095658.

The gene was expressed under the control of a triple promoter system (as described in WO 99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including mRNA stabilizing sequence.

Subsequently a recombinant Bacillus subtilis clone containing the integrated expression construct was grown in liquid culture. The culture broth was centrifuged (20000×g, 20 min) and the supernatant was carefully decanted from the precipitate and used for purification of the enzyme or alternatively sterile filtered supernatant was used directly for assays.

### Purification of the Recombinant Enzyme by Nickel Affinity Chromatography

The pH of the cleared supernatant was adjusted to pH 8, filtered through a 0.2 μM filter, and the supernatant applied to a 5 ml HisTrap™ excel column. Prior to loading, the column had been equilibrated in 5 column volumes (CV) of 50 mM Tris/HCl pH 8. In order to remove unbound material, the column was washed with 8 CV of 50 mM Tris/HCl pH 8, and elution of the target was obtained with 50 mM HEPES pH 7+10 mM imidazole. The eluted protein was desalted on a HiPrep™ 26/10 desalting column, equilibrated using 3 CV of 50 mM HEPES pH 7+100 mM NaCl. This buffer was also used for elution of the target, and the flow rate was 10 ml/min. Relevant fractions were selected and pooled based on the chromatogram and SDS-PAGE analysis.

### Example 2

### MISWA96 Wash Assay

### Preparation of Biofilm Wwatches

Biofilm swatches (10 cm×10 cm) were made by growing Brevundimonas sp. on polyester swatches for three days. The biofilm swatches were rinsed twice in water and dried for 2 h in a laf bench with flow and subsequently punched into round swatches circles (0.6 cm×0.6 cm) and placed into the wells of a MTP96 and stored at 4° C. for further use.

### Washing Experiment

The swatches were transferred from the MTP96 to a deep-well MTP96, for the washing assay. The deep-well plate was placed in a Hamilton robot and subjected to a wash simulation program using the following conditions: shaking speed: 30 sec at 1000 rpm. Duration of wash cycle: 30 minutes with shaking; temperature 30° C.; Volume of wash liquor (total): 500 μl per well (490 μl wash liquor of model detergent A+10 μl sample). For screening of wash performance of WT proteases, wash liquor model detergent A was prepared by dissolving 3.3 g/L in water hardness 15° dH. Soil was subsequently added to reach a concentration of 0.7 g soil/L (WFK 09V pigment soil).

A 96 deep-well plate was filled with each enzyme sample, and the program was started on the robot. The proteases were tested in concentration 0.5 ppm. The blank consisted of biofilm swatches without any enzyme addition. After completion of the wash simulation cycle, the swatches were removed from the wash liquor and dried on a filter paper. The dried swatches were fixed on a sheet of white paper for scanning. The scanned picture was further used with the software color analyzer. Each sample has an intensity measurement from the color analyzer software analysis that will be used to calculate the delta intensity (remission), by subtracting the intensity of the blank without enzyme. Values over 20 are visual for the human eye.

TABLE 1
Wash performance of the proteases.

| | Intensity | Intensity | |
|---|---|---|---|
| | (with | (without | Delta |
| Name | protease) | protease) | Intensity |
| SEQ ID NO: 21 | 233 | 179 | 54 |
| SEQ ID NO: 12 | 190 | 178 | 12 |
| SEQ ID NO: 6 | 222 | 200 | 22 |
| SEQ ID NO: 18 | 218 | 200 | 18 |

1. An isolated or purified polypeptide having proteolytic activity, selected from the group consisting of:
(a)
i. a polypeptide having at least 96%, at least 97%, at least 98%, at least 99% or at least 100% sequence identity to SEQ ID NO: 21,
ii. a polypeptide having at least 92%, at least 93%, at least 94%, at least 95%, at least 96%, at least 97%, at least 98%, at least 99% or at least 100% sequence identity to SEQ ID NO: 3,
iii. a polypeptide having at least 94%, at least 95%, at least 96%, at least 97%, at least 98%, at least 99% or at least 100% sequence identity to SEQ ID NO: 6,
iv. a polypeptide having at least 81%, at least 85%, at least 90%, at least 95%, at least 96%, at least 97%, at least 98%, at least 99% or at least 100% sequence identity to SEQ ID NO: 9,
v. a polypeptide having at least 60%, at least 65%, at least 70%, at least 75, at least 80%, at least 85%, at least 90%, at least 95%, at least 96%, at least 97%, at least 98%, at least 99% or at least 100% sequence identity to SEQ ID NO: 12, or
vi. a polypeptide having at least 55%, at least 60%, at least 65%, at least 70%, at least 75, at least 80%, at least 85%, at least 90%, at least 95%, at least 96%, at least 97%, at least 98%, at least 99% or at least 100% sequence identity to SEQ ID NO: 18,
(b) a polypeptide encoded by a polynucleotide that hybridizes under high stringency conditions with the full-length complement of the mature polypeptide coding sequence of SEQ ID NO: 19, 1, 4, 7, 10, 13 or 16;
(c) a polypeptide derived from a polypeptide of SEQ ID NO: 21, 3, 6, 9, 12, 15 or 18 by substitution, deletion or addition of one or several amino acids in the polypeptide of SEQ ID NO: 21, 3, 6, 9, 12, 15 or 18; and
(d) a fragment of the polypeptide of (a), (b) or (c) that has proteolytic activity.

2. The polypeptide of claim 1, comprising, consisting essentially of, or consisting of SEQ ID NO: 20 or a mature polypeptide thereof; or SEQ ID NO: 21; comprising, consisting essentially of, or consisting of SEQ ID NO: 2 or a mature polypeptide thereof or SEQ ID NO: 3; comprising, consisting essentially of, or consisting of SEQ ID NO: 5 or a mature polypeptide thereof or SEQ ID NO: 6; comprising, consisting essentially of, or consisting of SEQ ID NO: 8 or a mature polypeptide thereof or SEQ ID NO: 9; comprising, consisting essentially of, or consisting of SEQ ID NO: 11 or a mature polypeptide thereof or SEQ ID NO: 12; comprising, consisting essentially of, or consisting of SEQ ID NO: 14 or a mature polypeptide thereof or SEQ ID NO: 15; or comprising, consisting essentially of, or consisting of SEQ ID NO: 17 or a mature polypeptide thereof or SEQ ID NO: 18.

3. A granule, which comprises:
(a) a core comprising the polypeptide of claim 1 and;
(b) a coating consisting of one or more layer(s) surrounding the core.

4. A granule, which comprises:
(a) a core, and
(b) a coating consisting of one or more layer(s) surrounding the core, wherein the coating comprises the polypeptide of claim 1.

5. A composition comprising the polypeptide of claim 1.

6. A composition of claim 5, wherein the composition is a cleaning composition comprising at least one cleaning component.

7. The composition according to claim 5, wherein the cleaning component is a surfactant and/or a builder.

8. A cleaning composition comprising:
a) polypeptide selected from the group consisting of SEQ ID NO: 21, SEQ ID NO: 3, SEQ ID NO: 6, SEQ ID NO: 9, SEQ ID NO: 12, SEQ ID NO: 15 and SEQ ID NO: 18, and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity to any of said sequences, wherein the polypeptide has proteolytic activity, and
b) at least one surfactant.

9. A whole broth formulation or cell culture composition comprising the polypeptide of claim 1.

10. An isolated or purified polynucleotide encoding the polypeptide of claim 1.

11. A nucleic acid construct or expression vector comprising the polynucleotide of claim 10, wherein the polynucleotide is operably linked to one or more control sequences that direct production of the polypeptide in an expression host.

12. A recombinant host cell comprising the polynucleotide of claim 11 operably linked to one or more control sequences that direct production of the polypeptide.

13. A method of producing a polypeptide having proteolytic activity, comprising cultivating the recombinant host cell of claim 12 under conditions conducive for production of the polypeptide, and optionally recovering the polypeptide.

14. (canceled)

15. A method of cleaning an item, comprising the steps of:
a) exposing an item to a wash liquor comprising the composition of claim 5,
b) completing at least one wash cycle, and optionally
c) rinsing the item;
wherein the item is a textile or a hard surface.
